(19) United States
(12) Patent Application Publication
(10) Pub. No.: US 2025/0268876 A1
(43) Pub. Date: Aug. 28, 2025

(54) TREATMENT OF HIDRADENITIS SUPPURATIVA WITH ORISMILAST

(71) Applicant: UNION THERAPEUTICS A/S, Hellerup (DK)

(72) Inventors: Morten SOMMER, Hellerup (DK); Kim KJØLLER, Hellerup (DK); Philippe ANDRES, Peymeinade (FR); Anne WEISS, Hellerup (DK); Elisabeth Hjardem TAUDORF, Roskilde (DK); Gregor B.E JEMEC, Roskilde (DK)

(21) Appl. No.: 18/551,157

(22) PCT Filed: Mar. 22, 2022
(86) PCT No.: PCT/EP2022/057472

§ 371 Date: Sep. 18, 2023

(30) Foreign Application Priority Data
Mar. 22, 2021 (GB) 2103975.5
Dec. 17, 2021 (GB) 2118420.5
Feb. 3, 2022 (GB) 2201381.7

(51) Int. Cl. A61K 31/4436; A61K 9/20; A61K 9/28; A61K 45/06; A61P 17/10; A61P 29/00

(52) U.S. Cl. CPC A61K 31/4436; A61K 9/2009; A61K 9/2013; A61K 9/2018; A61K 9/2054; A61K 9/2813; A61K 9/284; A61K 45/06; A61P 17/10; A61P 29/00

(57) ABSTRACT

A compound of formula (I), or a pharmaceutically acceptable salt, solvate or hydrate thereof, for use in the treatment of hidradenitis suppurativa (HS) in a subject.

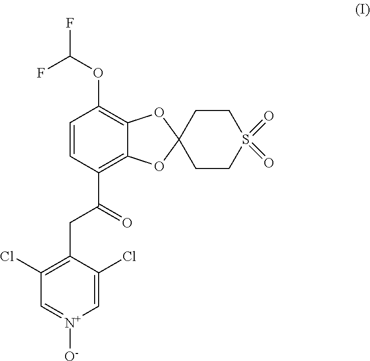 [SMILES: O=C(Cc1c(Cl)c[n+]([O-])cc1Cl)c1ccc(OC(F)F)c2c1OC1(CCS(=O)(=O)CC1)O2]

This invention relates to the use of a PDE4 inhibitor, in particular orismilast, in the treatment of one or more clinical signs or symptoms of Hidradenitis suppurativa (HS), in a subject.

### BACKGROUND

Hidradenitis suppurativa (HS), also known as acne inversa, is a chronic, recurrent, and debilitating skin condition (Jemec G, N Engl J Med. 2012, 366(2):158-64). It is an inflammatory disorder of the follicular epithelium and commonly occurs in the axillae, inframammary folds, and groin. HS typically presents with inflammatory nodules, abscesses, comedones, sinus tracts, or scarring. It has an insidious onset, starting with mild discomfort, erythema, burning, pruritus, and hyperhidrosis. It progresses to form tender or deep-seated nodules, which expand and coalesce to form large painful abscesses. The rupture of these abscesses releases malodorous and purulent discharge. Recurrent or persistent HS results in the formation of double-ended comedones, sinus tracts, and scarring. Secondary bacterial infection can often occur.

Complications of HS are distressing. Locally, HS can cause scarring with associated restricted limb mobility, strictures or fistulas at the anus and urethra from chronic inflammation, and disfigurement. Rarely, squamous cell carcinoma at the site of HS has also been reported. Systemically, HS with serious infection can present with fever and septicemia. Anemia is also associated with HS.

Early lesions of HS mimic other skin conditions and thus are often misdiagnosed as recurrent furunculosis or boils. The delay in HS diagnosis can be 12 years or longer. The diagnosis is made clinically based on typical lesions (nodules, abscesses, sinus tracts), locations (skin folds), and nature of relapses and chronicity. Multiple comorbidities are associated with HS, including obesity, metabolic syndrome, inflammatory bowel disease, and spondyloarthropathy. Although there are effective symptomatic management options, the lack of curative therapy and the recurrent nature makes HS treatment challenging.

Prompt recognition and initiation of treatment can reduce the risk of HS progression to debilitating end-stage disease. The psychosocial effect of HS is devastating because of the associated pain, malodorous discharge, and scarring. In fact, HS is associated with greater impairment of quality of life and professional activity than other chronic skin conditions such as psoriasis and atopic dermatitis.

The prevalence of HS is about 1% to 4% in Europe but lower in North America. Hidradenitis suppurativa is more common in females, with a female-to-male ratio of 4:1. The age of onset is usually after puberty and before the age of 40, peaking in the second and third decades of life. Although HS in prepubertal children is rare, the earliest reported age of onset is 6 years old.

The cause of HS is unclear but likely multifactorial. Genetics play a role, as up to 40% of patients with HS have a positive family history, but there has been no monozygotic concordance study for HS. An autosomal dominant mode of inheritance in HS is rare but has been reported.

Several exogenous factors are associated with HS. Hormones play a role given its post-pubertal and premenopausal pattern of onset. Specifically, androgen-containing medications precipitate or worsen HS, and antiandrogenic agents are used to treat HS. In addition, obesity can aggravate the disease, likely owing to the increased mechanical stress on the intertriginous skin. Obesity is also more commonly seen in HS, but the correlation between body mass index and HS severity is controversial. Finally, the association between cigarette smoking and HS is well documented, with a higher incidence of HS in smokers than non-smokers.

The general approach to HS treatment includes non-medical interventions, topical and systemic medications, and surgery. Some interventions are available to control the disease and improve symptoms. Goals of HS treatment include preventing new lesions, treating newly formed lesions early and effectively, and removing existing nodules and sinus tracts.

Many medications, such as topical resorcinol, oral and topical antibiotics, hormonal therapy, retinoids and immunosuppressive agents, are used to treat HS by preventing new lesions, removing existing nodules and sinus tracts, and improving symptoms. However, some existing medications have limited efficacy, are associated with a high recurrence rate and often have significant side-effects, as reported in case series and reports, RCTs, and retrospective studies (Lee Y E et al., Canadian Family Physician February 2017, 63 (2) 114-120).

Biologic agents with anti-TNF-α (tumour necrosis factor-α) properties, infliximab and adalimumab, have been demonstrated to be efficacious (see review by Lim S Y D and Oon H., Biologics. 2019 13:53-78). Adalimumab has been recently approved by the US Food and Drug Administration, as well as the European Medicines Agency, for the treatment of moderate to severe HS.

Recently, in two phase II studies that enrolled patients with moderate HS, a treatment with phosphodiesterase 4 (PDE4) inhibitor, apremilast, which is an approved drug for the treatment of psoriasis, showed that more than 50% of the treated patients had a positive HS clinical response (HiSCR) versus none of the patients in the placebo group (Vossen ARJV et al., J Am Acad Dermatol. 2019 80(1):80-88; Kerdel F R et al., J Drugs Dermatol. 2019 18(2):170-176). However, treatment of patients with apremilast was associated with adverse effects, primarily gastrointestinal (GI) side effects such as nausea, diarrhoea, and emesis, which are typical for PDE4 inhibitors. Another PDE4 inhibitor, Roflumilast, which is approved for the treatment of chronic obstructive pulmonary disease (COPD), has also been found to present tolerability issues, primarily GI effects including nausea, vomiting and diarrhoea.

Phosphodiesterases (PDEs) are the only enzymes that hydrolyze and degrade cAMP. PDE4 is a cAMP phosphodiesterase widely expressed in hematopoietic cells (e.g. myeloid, lymphoid), nonhematopoietic cells (e.g. smooth muscle, keratinocyte, endothelial), and sensory/memory neurons. The four PDE4 genes (A, B, C, and D) exhibit distinct target and regulatory properties. Each of these genes can produce multiple protein products due to mRNA splice variants, resulting in approximately 19 different PDE4 proteins that fall into either short or long isoform categories. Long isoforms are differentiated from short isoforms by an additional upstream conserved region (UCR), which contains a PKA activation site. These UCR sequences play a critical role in the regulation of PDE4 through the phosphorylation of PKA and extracellular signal-regulated kinase (ERK). For example, the major PDE4 isoforms expressed in leukocytes are PDE4 B2 (short isoform) and PDE4 D3 and D5 (long isoforms). Long PDE4 D isoforms predominate in monocytes, whereas short PDE4 B isoforms predominate in macrophages. The catalytic activity of PDE4 B2 is activated by ERK phosphorylation, whereas the catalytic function of the D3 and D5 variants is inhibited by ERK activation. Thus, the UCR modules can determine the functional outcome of ERK phosphorylation. This means that the pro-inflammatory mediators of monocytes trigger an overall decrease in PDE4 activity, whereas the proinflammatory mediators of macrophages trigger an overall increase in PDE4 activity.

As cAMP is a key second messenger in the modulation of inflammatory responses, PDE4 has been found to regulate inflammatory responses of inflammatory cells by modulating proinflammatory cytokines such as TNF-α, IL-2, IFN-γ, GM-CSF and LTB4. Inhibition of PDE4 has therefore become an attractive target for the therapy of inflammatory diseases, although it is associated with significant side effects, particularly nausea and emesis (Lagente V et al., Mem Inst Oswaldo Cruz, Rio de Janeiro, 2005 v.100(Suppl. I): 131-136; Shett G et al., Ther Adv Musculoskel Dis, 2010, v 2(5) 271-278).

HS is difficult to treat and no cure exists for this condition. Medical interventions that are both therapeutically efficacious and safe are therefore highly desirable. There remains a need for effective treatments for HS.

### BRIEF SUMMARY OF THE DISCLOSURE

According to a first aspect of the invention, there is provided a compound of formula (I):

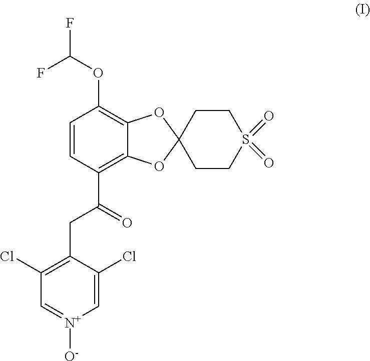 [SMILES: O=C(Cc1c(Cl)c[n+]([O-])cc1Cl)c1ccc(OC(F)F)c2c1OC1(CCS(=O)(=O)CC1)O2]

or a pharmaceutically acceptable salt, solvate or hydrate thereof for use in the treatment of hidradenitis suppurativa (HS) in a subject. The compound of formula (I) is 2-(3,5-Dichloro-1-oxido-pyridine-4-yl)-1-(7-difluoromethoxy-2′,3′,5′,6′-tetrahydro-spiro[1,3-benzodioxole-2,4′-(4H)-thiopyran-1′,1′-dioxide]-4-yl)ethenone, which is also known as orismilast.

In some embodiments, the treatment comprises the use of a pharmaceutically acceptable salt of the compound of formula (I).

According to a second aspect of the invention, there is provided a compound of formula (I) as defined herein, or a pharmaceutically acceptable salt, solvate or hydrate thereof, for use in preventing the progression of disease in a subject with mild or moderate HS.

According to a third aspect of the invention, there is provided a compound of formula (I) as defined herein, or a pharmaceutically acceptable salt, solvate or hydrate thereof, for use in reducing inflammation caused by or associated with HS.

The treatment may comprise oral, topical and/or intravenous administration of the compound of formula (I), or a pharmaceutically acceptable salt, solvate or hydrate thereof. In some embodiments the treatment comprises oral administration. In some embodiments the treatment comprises topical administration. In some embodiments the treatment comprises oral and topical administration.

The treatment may be administered for at least 10 days, at least 2 weeks, at least 3 weeks, at least 4 weeks, at least 6 weeks or at least 8 weeks. In some embodiments, the treatment is administered for no more than 20 weeks, no more than 16 weeks, no more than 12 weeks or no more than 10 weeks.

The treatment may comprise administering a total daily dose of no more than 120 mg, no more than 100 mg, no more than 80 mg, no more than 60 mg, or no more than 40 mg of the compound of formula (I). In some embodiments, the treatment comprises administering a total daily dose of at least 20 mg, at least 30 mg, at least 40 mg, at least 50 mg or at least 60 mg. For example, the total daily dose administered may be from about 5 to about 120 mg, from about 10 to about 120 mg, from about 20 to about 110 mg, from about 30 to about 100 mg, from about 40 to about 90 mg, from about 50 to about 80 mg, or from about 60 to about 70 mg. In some embodiments the total daily dose of the compound of formula (I) administered is from about 50 to about 90 mg, from about 60 to about 80 mg. In some embodiments the treatment comprises administering a total daily dose of about 40 mg of the compound of formula (I). In some embodiments the treatment comprises administering a total daily dose of about 10 mg of the compound of formula (I). In some embodiments the treatment comprises administering a total daily dose of about 20 mg of the compound of formula (I). In some embodiments the treatment comprises administering a total daily dose of about 60 mg of the compound of formula (I). In some embodiments the treatment comprises administering a total daily dose of about 80 mg of the compound of formula (I). In some embodiments the treatment comprises administering a total daily dose of about 100 mg of the compound of formula (I).

In some embodiments, the treatment comprises administering a dose of from about 10 to about 60 mg, from about 20 to about 50 mg, or from about 30 to about 40 mg.

The treatment may be administered from one to four times daily, for example from two to three times daily. In some embodiments the treatment is administered once daily. In some embodiments the treatment is administered twice daily.

The compound of formula (I) as defined herein, or a pharmaceutically acceptable salt, solvate or hydrate thereof, may be comprised within a composition or formulation. Thus, the invention also provides a composition or formulation comprising a compound of formula (I), or a pharmaceutically acceptable salt, solvate or hydrate thereof, for use in the treatment of HS. The composition or formulation may be formulated according to the desired route of administration.

In some embodiments, the compound, or a composition or formulation comprising the compound, is formulated for oral administration. Suitably, the compound, composition or formulation is formulated in the form or a tablet or capsule. In some embodiments, the compound is comprised within a modified-release formulation.

The subject may be a human or an animal. In some embodiments the subject is a human. In some embodiments the subject is a human male. In some embodiments the subject is a human female.

The subject may have mild, moderate or severe HS. In some embodiments, the subject has mild HS. In some embodiments, the subject has moderate HS. In some embodiments, the subject has severe HS.

In some embodiments, the subject is suffering from a comorbidity selected from obesity, metabolic syndrome, inflammatory bowel disease, spondyloarthropathy, or any combination thereof.

In some embodiments, the subject has not been previously treated with an antibody or other biological therapy for HS. In some embodiments, the subject has not been previously treated with a TNF-α inhibitor (e.g. adalimumab).

In other embodiments, the subject has been previously treated with an antibody or other biological therapy for HS. In some embodiments the subject has been previously been treated with an anti-inflammatory antibody. In some embodiments, the subject has been previously treated with a TNF-α inhibitor (e.g. adalimumab). It may be that the subject is non-responsive or refractory to prior treatment of the HS with an antibody therapy, for example where the subject is non-responsive or refractory to treatment with a TNF-α inhibitor (e.g. adalimumab).

The treatment may be administered in combination with a further therapy. The further therapy may be selected from an anti-androgenic agent, a hormone, an antibiotic, a retinoid, an anti-inflammatory agent (including steroids and non-steroidal anti-inflammatory agents), an analgesic, an immunosuppressive agent, an antibody, surgery or any combination thereof.

In some embodiments the treatment provides selective inhibition of PDE4D and/or PDE4B. In certain embodiments the treatment provides selective inhibition of PDE4B2, PDE4B3, PDE4D2, PDE4D3, PDE4D4, PDE4D5 and/or PDE4D7. In further embodiments, the treatment may provide selective inhibition of PDE4D3 and/or PDE4B2.

### DETAILED DESCRIPTION

The invention will now be described by way of example and with reference to the accompanying Figures, in which:

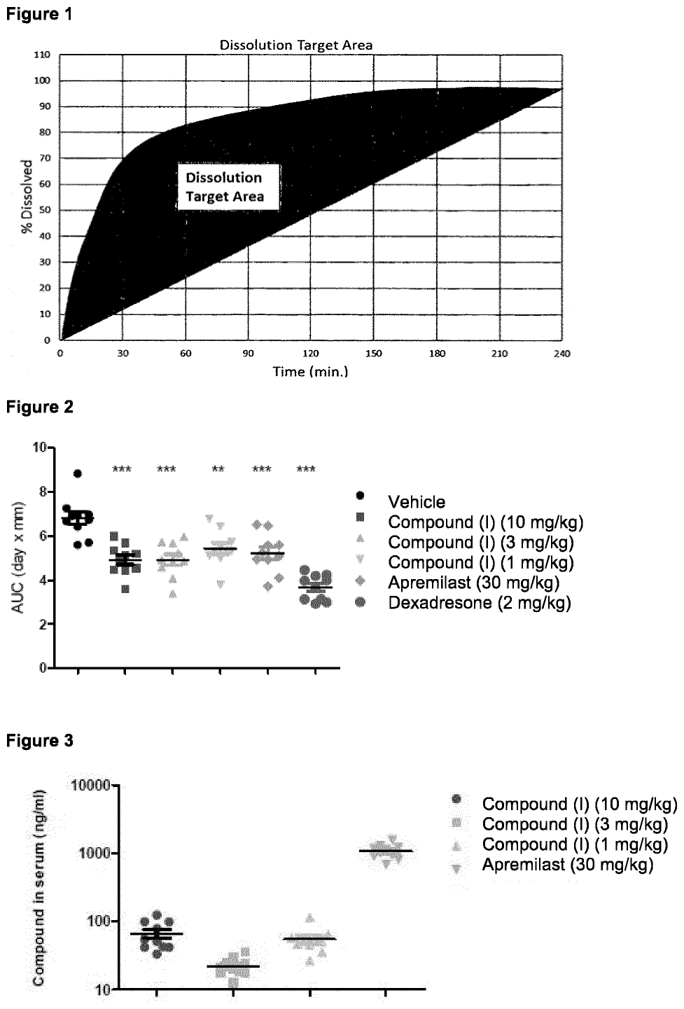

FIG. 1 is a chart illustrating the dissolution target area for a modified release formulation; dissolution method: Paddle 75 rpm, 900 ml 0.1N HCl+0.5% SDS, 37° C., HPLC detection;

FIG. 2 shows the AUC for ear thickness in mice dosed with the compound of formula (I) or apremilast; and

FIG. 3 shows the concentration of compound in serum in mice dosed with the compound of formula (I) or apremilast.

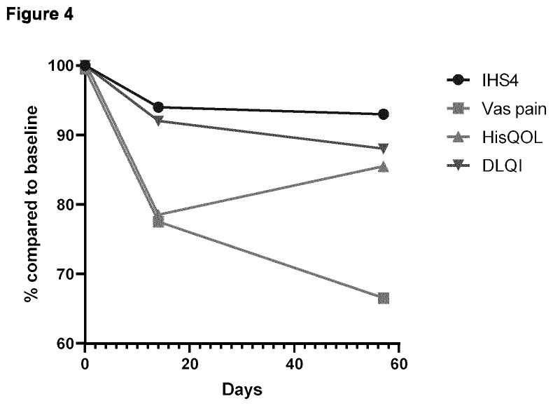

FIG. 4 shows the % change from baseline during the course of the treatment of the subject in Example 9 with the compound of formula (I). This figure shows the % change relative to baseline in international HS severity score (IHS4), Visual analog scale of pain (VAS pain), HS specific quality of life (HiSQOL) and Dermatology Life Quality Index (DLQI).

### DEFINITIONS

Unless otherwise stated, the following terms used in the specification and claims have the following meanings set out below.

The terms “treating” or “treatment” refers to any indicia of success in the treatment or amelioration of a disease, pathology or condition, including any objective or subjective parameter such as abatement; remission; diminishing of symptoms or making the pathology or condition more tolerable to the subject; slowing in the rate of degeneration or decline; making the final point of degeneration less debilitating; improving the physical or mental well-being of the subject. For example, in relation to HS, “treatment” may comprise one or more of: eliminating, or reducing the severity, spread and/or number of, inflammatory nodules, abscesses, comedones and/or sinus tracts; reducing or eliminating pain, inflammation, burning, swelling, redness, itching and/or discomfort; reducing or eliminating discharge; preventing or reducing scarring and/or disfigurement; preventing or reducing the likelihood and/or severity of secondary infection (e.g. bacterial infection), cancer (e.g. squamous cell carcinoma), septicaemia and/or anaemia; preventing or minimizing the incidence of depression and other psychological effects; preventing or delaying the progression of disease from mild to moderate or from moderate to severe HS; a reduction in the disease severity according to the Physician's Global Assessment of disease severity (HS-PGA); and an improvement in the subject's score according to the Hidradenitis Suppurati, the Dermatology quality of Life Index (DLQI), the European quality of life-5 Dimensions (EQ-5D) scale, the international HS severity score (IHS4), HS specific quality of life (HiSQOL), the Dermatology Life Quality Index (DLQI), the McGill Pain questionnaire, the Visual analog scale of pain (VAS pain), the Work Productivity and Activity Impairment Questionnaire: Specific Health Problem V2.0 (WPAI:SHP), the Anxiety and depression (HADS) questionnaire and/or the Multidimensional Fatigue Inventory 20 (MFI-20). The compound of formula (I) has a rapid onset of action and may therefore provide rapid relief of one or more symptoms and/or clinical features of HS. For example administration of the compound of formula (I) to a subject with HS may provide rapid relief of pain associated with HS.

When a compound or salt, solvate or hydrate described in this specification is administered to treat a disorder, a “therapeutically effective amount” is an amount sufficient to reduce or completely alleviate symptoms or other detrimental effects of the disorder; cure the disorder; reverse, completely stop, or slow the progress of the disorder; or reduce the risk of the disorder getting worse.

The term “pharmaceutically acceptable salt” refers to salts that retain the biological effectiveness and properties of the compounds described herein, and which are not biologically or otherwise undesirable. Pharmaceutically acceptable salts of compound (I) are well known to skilled persons in the art. It may be that a reference to a salt of compound (I) herein may refer to a pharmaceutically acceptable salt of compound (I). The invention covers all crystalline modifications, polymorphic forms and mixtures thereof. In some embodiments, the treatment comprises administration of the polymorphic form E of the compound of formula (I).

The term “solvate” is intended to indicate a species formed by interaction between a compound, e.g. a compound of formula (I), and a solvent, e.g. alcohol, glycerol or water, wherein said species are in a solid form. When water is the solvent, said species is referred to as a “hydrate”.

The phrase “phosphodiesterase” as used herein refers to one or more of the phosphodiesterases (PDEs), PDE4, PDE7 and PDE8 being selective for cAMP. PDE4 is the most important modulator of cAMP. PDE4 is cAMP-specific and the dominant PDE in inflammatory cells. PDE4 enzymes are encoded by four genes (PDE4A, PDE4B, PDE4C and PDE4D), each of which is capable of producing a number of isoforms through mRNA splicing and the use of different promoters. Each PDE4 isoform within a particular PDE4 sub-family comprises a common core region, consisting of the catalytic unit and the C-terminal portion, and is defined by its unique N-terminal region. The PDE4 isoforms are further classified as long, short or super-short, depending on the presence or absence (or truncation) of two highly conserved sequences: Upstream Conserved Region 1 (UCR1) and Upstream Conserved Region 2 (UCR2). “Long” isoforms comprise both UCR1 and UCR2; “short” isoforms lack UCR1 and “super-short” isoforms lack UCR1 and have a truncated UCR2.

The phrase “PDE inhibitor” as used herein refers to a substance which inhibits PDE.

References to “topical treatment” or “topical administration” refer to the application of the compound, or a formulation comprising the compound, to the skin, soft tissues or mucous membranes.

Reference to a “subject” herein means a human or animal subject. Preferably the subject is warm-blooded mammal. More preferably the subject is a human.

Unless stated otherwise, reference herein to a “% by weight of the compound of formula (I), or a pharmaceutically acceptable salt, solvate or hydrate thereof” is intended to refer to the amount of the non-salt form of the compound. For example, reference to a composition comprising “5% by weight of the compound of formula (I) or a pharmaceutically acceptable salt, solvate or hydrate thereof” refers to a composition comprising 5% by weight of the compound in the non-salt form. Accordingly, where such a composition comprises a pharmaceutically acceptable salt the compound, the absolute amount of the salt in the composition will be higher than 5% by weight in view of the salt counter ion that will be also be present in the composition.

Reference to “about” in the context of a numerical is intended to encompass the value+/−10%. For example, about 20% includes the range of from 18% to 22%.

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

### Compound of Formula (I)

The present invention relates to a formulation comprising a compound of formula (I):

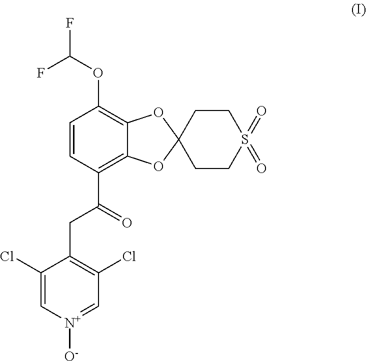 [SMILES: O=C(Cc1c(Cl)c[n+]([O-])cc1Cl)c1ccc(OC(F)F)c2c1OC1(CCS(=O)(=O)CC1)O2]

or a pharmaceutically acceptable salt, solvate or hydrate thereof.

The compound of formula (I), 2-(3,5-Dichloro-1-oxido-pyridine-4-yl)-1-(7-difluoro-methoxy-2′,3′,5′,6′-tetrahydro-spiro[1,3-benzodioxole-2,4′-(4H)-thiopyran-1′,1′-dioxide]-4-yl)ethanone, also referred to herein as “orismilast”, was disclosed in WO 2011/160632, relating to benzodioxole and benzodioxepene heterocyclic compounds useful as PDE4 inhibitors for use in the treatment, prevention or alleviation of a variety of diseases, such as dermal diseases or conditions, such as proliferative and inflammatory skin disorders, dermatitis, psoriasis, psoriasis vulgaris, atopic dermatitis, seborrheic dermatitis, contact dermatitis, cancer, epidermal inflammation, alopecia, alopecia areata, skin atrophy, steroid induced skin atrophy, skin ageing, photo skin ageing, acne, urticaria, pruritis, and eczema.

The compound of formula (I) should be understood to include any pharmaceutically acceptable form and salts, hydrates or solvate of the compound. The compound may be present in a crystalline or amorphous form. The compound of formula (I) is considered as being a poorly soluble compound. The compound of formula (I) and salts thereof, and methods for synthesizing the compound, are disclosed in WO 2011/160632, WO 2015/197534, WO 2017/103058, and WO 2018/234299.

In any of the aspects of the invention, the compound of formula (I) may be present in a crystalline form. For example, the compound of formula (I) may be the polymorphic Form E of the compound. Form E of the compound of formula (I) is described in WO 2018/234299 and has an XRPD diffractogram pattern substantially as shown in FIG. 1 of WO 2018/234299, which is incorporated herein by reference thereto. Form E is characterised as having a melting endotherm with an onset temperature of about 217° C. to about 219° C. when measured by DSC with a heating rate of 100° C./minute under a nitrogen atmosphere. Form E may be prepared by crystallisation of a compound of formula (I) from a suitable solvent, for example ethanol or acetone.

Orismilast (the compound of formula (I)) is a selective and efficient inhibitor of PDE4. It has been found that orismilast is a selective inhibitor of PDE4D and PDE4B. In some embodiments orismilast is a selective inhibitor of PDE4B1, PDE4B2, PDE4B3, PDE4D1, PDE4D2, PDE4D3, PDE4D4, PDE4D5 and/or PDE4D7. In some embodiments orismilast is a selective inhibitor of PDE4B2, PDE4B3, PDE4D2, PDE4D3, PDE4D4, PDE4D5 and/or PDE4D7. In some embodiments orismilast is a selective inhibitor of PDE4B2, PDE4B3, PDE4D5 and PDE4D7. In some embodiments orismilast is a selective inhibitor of PDE4B3, PDE4D5 and PDE4D7. In particular, it has been found that orismilast is a selective inhibitor of the PDE4 isoforms PDE4D3 and PDE4B2. In addition, orismilast has been found to potently inhibit the secretion of TNF-α and IL-1β, two cytokines that are highly associated with inflammation. The compound also inhibits IFN-γ. IFN-γ is a T-cell derived Th1 cytokine that plays a role in Th1 immune responses. The ability of orismilast to inhibit cytokines involved in inflammation, particularly those which are linked to HS, supports the use of orismilast in the treatment of HS. Significantly, orismilast has been shown to an average of 23 times more potent than apremilast on a molar basis, in both LPS and SEB-induced TNF-α secretion from human whole blood.

### Pharmaceutical Compositions

The compound of formula (I), or the pharmaceutically acceptable salt, solvate or hydrate thereof may be comprised within a formulation. Thus, the present invention also provides a formulation, or a composition, comprising the compound of formula (I), or the pharmaceutically acceptable salt, solvate or hydrate thereof, for use in the treatment of HS. Compositions and formulations may be prepared according to, for example, the desired mode of administration e.g. oral, topical or intravenous administration.

Compositions and formulations comprising the compound may optionally further comprise one or more viscosity modifying agents, carriers (e.g. mannitol, lactose, microcrystalline cellulose or trehalose), emulsifiers, surfactants, humectants, oils, waxes, polymers, preservatives, pH modifying agents (for example a suitable acid or base, for example an organic acid or organic amine base), buffers, stabilizers, electrolytes antioxidants (for example, butylated hydroxyanisol or butylated hydroxytoluene), crystallisation inhibitors (for example a cellulose derivative such as hydroxypropylmethyl cellulose or polyvinylpyrrolidone), colorants, fragrances and taste-masking agents. Such excipients are well-known, for example as listed in the Handbook of Pharmaceutical Excipients, 7th Edition, Rowe et al.

In some embodiments the compound of formula I, or a pharmaceutically acceptable salt, solvate or hydrate thereof, is present in the formulation in an amount of from about 0.01 to 50% by weight of a solid formulation. Thus it may be that the compound of formula I, or a pharmaceutically acceptable salt, solvate or hydrate thereof, is present in the solid formulation in an amount of about 0.05 to 40%, from 0.1 to 30%, from 0.2 to 20%, from 0.3 to 15%, from 0.4 to 12%, from 0.5 to 11%, from 1 to 10%, from 1.5 to 9.5%, from 2 to 9%, from 2.5 to 8.5%, from 3 to 8%, from 3.5 to 7.5%, from 4 to 7%, from 4.5 to 6.5%, or from about 5 to 6%, e.g. about 5.5%.

### Formulations for Topical Administration

In some embodiments the compound is formulated for topical administration to the subject. For example, a formulation comprising the compound of formula (I), or a pharmaceutically acceptable salt, solvate or hydrate thereof, may be topically applied to the skin at a site affected by HS, for example by topically applying the formulation directly to a HS lesion. Formulations suitable for topical administration include liquid or semi-liquid preparations such as liniments, lotions, gels, sprays or foams; oil-in-water or water-in-oil emulsions such as creams, ointments or pastes; or solutions or suspensions such as drops or sprays.

For topical administration, the compound of formula I may typically be present in the formulation in an amount of from 0.01 to 20% by weight of the composition. For example, the compound may be present in the formulation in an amount of from 0.02 to 18%, from 0.03 to 15%, from 0.04 to 15%, from 0.05 to 10%, from 0.06 to 9%, from 0.07 to 8%, from 0.08 to 6%, from 0.09 to 5%, from 0.1 to 4.5%, from 0.15 to 4%, from 0.2 to 3.5%, from 0.3 to 3%, from 0.4 to 2.5%, from 0.5 to 2%, from 0.6 to 1.5% or from 0.7 to 1%, by weight of the composition In some embodiments, the compound may be present in the formulation in an amount of from 0.01 to 5%, or from 0.02 to 2%.

### Formulations for Oral Administration

Formulations comprising the compound of formula (I), or a pharmaceutically acceptable salt, solvate or hydrate thereof, suitable for oral administration may be in the form of discrete units such as capsules, sachets, tablets or lozenges, each containing a predetermined amount of the compound of formula (I). The discrete units may contain the formulation in the form of a powder or granules, a solution or a suspension in aqueous or non-aqueous liquid, such as ethanol or glycerol, or in the form of an oil-in-water emulsion or a water-in-oil emulsion. Such oils may be edible oils, such as e.g. cottonseed oil, sesame oil, coconut oil or peanut oil. Suitable dispersing or suspending agents for aqueous suspensions include synthetic or natural gums such as tragacanth, alginate, acacia, dextran, sodium carboxymethylcellulose, gelatin, methylcellulose, hydroxypropyl methylcellulose, hydroxypropylcellulose, carbomers and polyvinylpyrrolidone. The formulation may also be administered in the form of a bolus, electuary or paste.

Powders may be prepared using well-known methods, for example by milling, blending, micro-precipitation, lyophilisation or spray drying, or spray-freeze drying a solution comprising a compound of formula (I), or a pharmaceutically acceptable salt, solvate or hydrate thereof.

The amount of the compound of formula (I), or a pharmaceutically acceptable salt, solvate or hydrate thereof, in each oral dosage form (e.g. tablet, capsule, sachet or lozenge) may range from about 1 mg to about 100 mg. The amount of the compound may for example range from 5 mg to 80 mg, from 10 mg to 60 mg, from 15 mg to 50 mg, from 20 to 40 mg or from 25 to 30 mg. In some embodiments, the amount of the compound of formula I, or a pharmaceutically acceptable salt, solvate or hydrate thereof, in each oral dosage form (e.g. tablet, capsule, sachet or lozenge) may range from about 10 mg to about 40 mg, for example from about 10 to about 30 mg. In some embodiments, the amount of the compound of formula I, or a pharmaceutically acceptable salt, solvate or hydrate thereof, in each oral dosage form (e.g. tablet, capsule, sachet or lozenge) is 1 mg, 2 mg, 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg or 100 mg.

In certain embodiments particles comprising the compound of formula (I), or a pharmaceutically acceptable salt, solvate or hydrate thereof, may be prepared by precipitation, lyophilisation or spray drying, or spray-freeze drying a solution comprising the compound and a suitable carrier to provide powder particles comprising the compound of formula (I) and the carrier as composite particles. Suitable carriers include inert carriers such as starch, sugars (e.g. mannitol, lactose, microcrystalline cellulose or trehalose).

In some embodiments the compound of formula (I), or a pharmaceutically acceptable salt, solvate or hydrate thereof is present in the formulation as a micronised powder, for example a micronised crystalline powder (e.g. micronised crystalline form E of the compound of formula (I)). Thus it may be that the particle size distribution of the compound in the formulation has a D50≤25 μm, for example D50≤20 μm, D50≤10 μm, D50≤5 μm, or D50≤3 μm.

Powders comprising a compound of formula (I), or a pharmaceutically acceptable salt, solvate or hydrate thereof, as described herein, may be dissolved or suspended in a suitable solvent (preferably water) prior to administration, e.g. application of a spray or gel.

A tablet may be made by compressing or moulding the formulation, optionally with one or more accessory ingredients. Compressed tablets may be prepared by compressing, in a suitable machine, the formulation in a free-flowing form such as a powder or granules, optionally mixed by a binder, such as e.g. lactose, glucose, starch, gelatine, acacia gum, tragacanth gum, sodium alginate, carboxymethylcellulose, methylcellulose, hydroxypropyl methylcellulose, polyethylene glycol, waxes or the like; a lubricant such as sodium oleate, sodium stearate, magnesium stearate, sodium benzoate, sodium acetate, sodium chloride or the like; a disintegrating agent such as starch, methylcellulose, agar, bentonite, croscarmellose sodium, sodium starch glycollate, crospovidone or the like or a dispersing agent, such as polysorbate 80.

Moulded tablets may be made by moulding. Suitable techniques for moulding tablets are well-known in the art. For example, in a suitable machine, a mixture of the powdered active ingredient and suitable carrier may be moistened with an inert liquid diluent. In some embodiments, moulded tablets may be made by dispersing a water-soluble excipient with the powdered active ingredient in a suitable solvent such as water, alcohol or organic solvents (e.g. acetone, hydrocarbons). Alternatively, moulded tablets may be made using thermoplastic polymers (e.g. polyethyleneoxide or polyvinyl caprolactam-polyvinylacetate-polyethylene glycol copolymers), without an inert liquid diluent.

### Modified Release Formulation

In some embodiments, the compound of formula (I), or a pharmaceutically acceptable salt, solvate or hydrate thereof, is comprised within a modified release formulation, for example a modified release formulation for oral administration. Thus it may be that the compound may be comprised within a modified release tablet formulation for oral administration. The use of modified release formulations may control the release of the therapeutic agent and thus control drug absorption from gastrointestinal tract. It has previously been described by the Applicant (in a PCT application published as WO2020/148271) that beneficial effects with respect to improved tolerability towards gastrointestinal adverse events and maintained systemic exposure can be achieved by formulating a PDE4 inhibitor in a modified release tablet formulation, wherein the in-vitro release is fast in comparison to a typically modified release profile but not yet as fast as for an immediate release tablet where major tolerability issues were seen.

It will be appreciated that the rate of dissolution of a modified release formulation will be determined by several factors e.g. the type and quantity of hydrophilic matrix former, excipients (fillers and coating) and the particle size of the drug substance. The dissolution target area in FIG. 1 illustrates the optimal area. Preferably, the modified release formulation provides the release and dissolution of the compound of formula (I) within the optimal area. The dissolution profile of a given formulation can be determined using the methods described in WO2020/0148271.

In some embodiments the orismilast is formulated as a modified release formulation described in WO2020/0148271, which is incorporated herein by reference thereto.

In some embodiments the modified release formulation releases less than 40% of the compound of formula (I) after 12 minutes. In some embodiments the modified release formulation releases less than 40% of the compound of formula (I) after 30 minutes. In some embodiments the modified release formulation releases less than 35% of the compound of formula (I) after 30 minutes. In some embodiments the modified release formulation releases from about 20% to about 40% of the compound of formula (I) after 30 minutes. In some embodiments the modified release formulation releases from about 25% to about 35% of the compound of formula (I) after 30 minutes. In some embodiments the modified release formulation releases from about 11% to about 65% of the compound of formula (I) after 45 minutes. In some embodiments the modified release formulation releases from about 25% to about 60% of the compound of formula (I) after 45 minutes. In some embodiments the modified release formulation releases from about 30% to about 45% of the compound of formula (I) after 45 minutes. In some embodiments the modified release formulation releases from about 35% to about 55% of the compound of formula (I) after 60 minutes. In some embodiments the modified release formulation releases more than about 60% of the compound of formula (I) after 60 minutes. In some embodiments the modified release formulation releases from about 35% to about 50% of the compound of formula (I) after 60 minutes. In some embodiments the modified release formulation releases from about 40% to about 50% of the compound of formula (I) after 60 minutes. In some embodiments the modified release formulation releases from about 50% to about 60% of the compound of formula (I) after 90 minutes. In some embodiments the modified release formulation releases from about 60% to about 80% of the compound of formula (I) after 120 minutes. In some embodiments the modified release formulation releases from about 60% to about 70% of the compound of formula (I) after 120 minutes. In some embodiments the modified release formulation releases more than about 70% of the compound of formula (I) after 180 minutes. In some embodiments the modified release formulation releases more than about 80% of the compound of formula (I) after 180 minutes. In some embodiments the modified release formulation releases from about 70% to about 100% of the compound of formula (I) after 180 minutes. In some embodiments the modified release formulation releases from about 80% to about 100% of the compound of formula (I) after 180 minutes. In some embodiments the modified release formulation releases from about 85% to about 100% of the compound of formula (I) after 180 minutes. In some embodiments the modified release formulation releases from about 90% to about 100% of the compound of formula (I) after 180 minutes. In some embodiments the modified release formulation releases from about 95% to about 100% of the compound of formula (I) after 180 minutes. In certain embodiments the modified release formulation releases from about 11% to about 65% of the compound of formula (I) after 45 minutes and more than 80% of the compound of formula (I) after 180 minutes. In certain embodiments the modified release formulation releases from about 25% to about 65% of the compound of formula (I) after 45 minutes and at least 75% of the compound of formula (I) after 180 minutes. In certain embodiments the modified release formulation releases from about 30% to about 50% of the compound of formula (I) after 45 minutes and at least 75% of the compound of formula (I) after 180 minutes. In some embodiments the modified release formulation releases from about 30% to about 55% of the compound of formula (I) after 45 minutes and at least 85% of the compound of formula (I) after 180 minutes. In certain embodiments the modified release formulation releases from about 30% to about 50% of the compound of formula (I) after 45 minutes and about 80% to about 100% of the compound of formula (I) after 180 minutes. In some embodiments the modified release formulation releases about 50% of the compound of formula (I) between about 60 and 100 minutes. In some embodiments the modified release formulation releases about 80% of the compound of formula (I) between about 120 and 180 minutes. In some embodiments the modified release formulation releases less than 40% of the compound of formula (I) after 30 minutes; 50% of the compound of formula (I) is released between 60 and 100 minutes; and 80% of the compound of formula (I) is released between 115 and 180 minutes. The modified release composition may, for example, be any of the modified release compositions described herein. In some embodiments the modified release composition comprises a compound of the formula (I), or a pharmaceutically acceptable salt, solvate or hydrate thereof; and a pharmaceutically acceptable hydrophilic matrix former (e.g. HPMC). In some embodiments the modified release composition comprises a compound of the formula (I), or a pharmaceutically acceptable salt, solvate or hydrate thereof; a pharmaceutically acceptable hydrophilic matrix former (e.g. HPMC); and a filler (e.g. lactose monohydrate). In some embodiments the modified release composition comprises a compound of the formula (I), or a pharmaceutically acceptable salt, solvate or hydrate thereof; and 15% w/w to 30% w/w of a pharmaceutically acceptable hydrophilic matrix former (e.g. HPMC). In some embodiments the modified release composition comprises a compound of the formula (I), or a pharmaceutically acceptable salt, solvate or hydrate thereof; and 15% w/w to 25% w/w of HPMC.

In the paragraph above reference to “release of the compound of formula (I)” refers to the % by weight of the compound of formula (I) initially present in the modified release formulation that is released into a dissolution medium at the specified time point, wherein the dissolution is determined using Ph. Eur. 2.9.3 Apparatus II, with a dissolution medium of 900 ml 0.5% sodium dodecyl sulfate in 0.1N HCl and a paddle speed of 75 rpm. The amount of the compound of formula (I) present in the dissolution medium may be determined by reversed phase, isocratic HPLC using a C18 column and UV detection at 272 nm. Suitably the % release values of the compound of formula (I) is a mean value obtained by measuring the release profile of more than one sample of the modified release formulation, thereby reducing the effects of inter- or intra-batch variability. The mean release % may be obtained by measuring the release from, for example 6, 12 or 24 samples of the modified-release formulation.

In some embodiments, the modified release tablet formulation comprises:
(i) the compound of formula (I), or a pharmaceutically acceptable salt, solvate or hydrate thereof;
(ii) one or more of a pharmaceutically acceptable hydrophilic matrix former;
(iii) optionally, one or more pharmaceutically acceptable excipients selected from the group consisting fillers, glidants and lubricants; and
(iv) optionally a pharmaceutically acceptable coating system.

In some embodiments the hydrophilic matrix former in the modified release formulation comprises hydroxypropyl methylcellulose or hydroxypropylcellulose, or mixtures thereof.

In some embodiments the hydrophilic matrix former in the modified release formulation comprises hydroxypropyl methylcellulose (HPMC). In some embodiments the hydrophilic matrix former in the modified release formulation consists of HPMC. In some embodiments the HPMC has a viscosity of from 30 to 150 mPa·s. In some embodiments the HPMC has a viscosity of from 35 to 130 mPa·s. In some embodiments the HPMC has a viscosity of from 40 to 60 mPa·s. In some embodiments the HPMC has a viscosity of from 80 to 120 mPa·s. Reference herein to the viscosity of HPMC refers to the viscosity of a 2% (w/w) solution of the HPMC in water at 20° C. in accordance with United States Pharmacopoeia (USP XXIII).

In some embodiments the hydrophilic matrix former in the modified release formulation comprises HPMC with a methoxyl substitution of from about 5% to about 35% In some embodiments the HPMC has a methoxyl substitution of from about 15% to about 30%. In some embodiments the HPMC has a methoxyl substitution of from about 19% to about 24%. In some embodiments the HPMC has a methoxyl substitution of from about 25% to about 35%. In some embodiments the HPMC has a methoxyl substitution of from about 28% to about 30%.

In some embodiments the hydrophilic matrix former in the modified release formulation comprises HPMC with a hydroxypropyl substitution of from about 4% to about 15%. In some embodiments the hydrophilic matrix former in the modified release formulation comprises HPMC with a hydroxypropyl substitution of from about 4% to about 12%. In some embodiments the hydrophilic matrix former in the modified release formulation comprises HPMC with a hydroxypropyl substitution of from about 7% to about 12%.

In some embodiments the hydrophilic matrix former in the modified release formulation comprises HPMC with a methoxyl substitution of from about 19% to about 24%; and a hydroxypropyl substitution of from about 4% to about 12%.

In some embodiments the hydrophilic matrix former in the modified release formulation comprises HPMC with a methoxyl substitution of from about 19% to about 24%; and a hydroxypropyl substitution of from about 7% to about 12%.

In some embodiments the hydrophilic matrix former in the modified release formulation comprises HPMC with a methoxyl substitution of from about 28% to about 30%; and a hydroxypropyl substitution of from about 7% to about 12%.

In some embodiments the hydroxypropyl methylcellulose is Hypromellose 2910, hypromellose 2208, or mixtures thereof.

Suitably the hydrophilic matrix former is present in a concentration from about 10% w/w to about 30% w/w hydroxypropyl methylcellulose. Thus it may be that the hydrophilic matrix former is present in a concentration from about 15% w/w to about 25% w/w hydroxypropyl methylcellulose. For example, wherein the hydrophilic matrix former is present in a concentration of 17.5% w/w hydroxypropyl methylcellulose. The hydroxypropyl methylcellulose may be, for example, any of the grades of hydroxypropyl methylcellulose disclosed herein (e.g. Hypromellose 2910, Hypromellose 2208, or mixtures thereof).

In some embodiments the one or more pharmaceutically acceptable excipients present in the modified release formulation comprises a filler, selected from lactose monohydrate and microcrystalline cellulose, and mixtures thereof. In some embodiments the fillers are present in a concentration from about 30% to about 78% w/w of lactose monohydrate and from 0% to about 40% w/w of microcrystalline cellulose. In some embodiments the filler is lactose monohydrate. In some embodiments the filler is lactose monohydrate and is present in a concentration from about 30% to about 78% w/w. Thus it may be that the lactose monohydrate is present in a concentration of about 71% w/w.

In some embodiments the modified release formulation comprises a coating. For example a PVA-based coating system.

In some embodiments the modified release formulation comprises
(i) the compound of formula (I), or a pharmaceutically acceptable salt, solvate or hydrate thereof;
(ii) a hydrophilic matrix former, wherein the hydrophilic matrix former is present in a concentration of from about 15% w/w to about 25% w/w hydroxypropyl methylcellulose;
(iii) from about 30% w/w to about 78% w/w lactose monohydrate;
(iv) optionally one or more pharmaceutically acceptable excipients selected from the group consisting of glidants and lubricants; and
(v) optionally a pharmaceutically acceptable coating system.

In some embodiments the modified release formulation comprises the compound of formula (I), about 0.5% w/w colloidal silicon dioxide, about 1.0% w/w magnesium stearate; and optionally a PVA-based coating system.

In some embodiments the modified release formulation comprises the compound of formula (I), about 17.5% w/w hypromellose, about 71.0% w/w lactose monohydrate, about 0.5% w/w colloidal silicon dioxide, about 1.0% w/w magnesium stearate; and optionally a PVA-based coating system.

In some embodiments the compound of formula (I) is present in the modified release formulation in an amount of from about 1% w/w to about 40% w/w, for example about 1% w/w to about 30% w/w, from about 1% w/w to about 20% w/w, or from about 2% w/w to about 15% w/w. In some embodiments the compound of formula (I) is present in the modified release formulation in an amount of from about 2% w/w to about 5% w/w. In some embodiments the compound of formula (I) is present in the modified release formulation in an amount of from about 8% w/w to about 12% w/w.

In some embodiments the compound of formula (I) is present in the modified release formulation in an amount of from about 5 mg to about 60 mg; about 10 mg to about 50 mg. For example about 10 mg, or about 30 mg.

In some embodiments, the compound may be evenly distributed in the modified release tablet formulation. The compound may be micronized. In some embodiments, the compound is crystalline. In some embodiments, the compound is crystalline and micronized.

In some embodiments the formulation comprises polymorphic form E of the compound of formula (I). In some embodiments, the polymorphic form E of the compound is micronized. In some embodiments, the polymorphic form E of the compound is crystalline and micronized.

The hydrophilic matrix former may be hydroxypropyl methylcellulose (HPMC), hydroxypropylcellulose (HPC), or mixtures thereof. For example, the hydrophilic matrix former could be hydroxypropyl methylcellulose, and mixtures thereof. The hydrophilic matrix former may be present at various concentrations and combinations from about 10% w/w to about 30% w/w HPMC. In some embodiments the hydrophilic matrix former is present in an amount of from about 15% w/w to about 25% w/w HPMC. In some embodiments the hydrophilic matrix former is present in an amount of from about 15% w/w to about 20% w/w HPMC. In some embodiments the hydrophilic matrix former is present in an amount of 17.5% w/w HMPC.

In some embodiments the modified release tablet formulation comprises one or more fillers and/or binders. The term “filler” as used herein may also function as a binder. The filler or binder may be selected from lactose monohydrate, lactose hydrous or anhydrous, microcrystalline cellulose, mannitol, isomalt, and mixtures thereof. For example, the filler could be lactose monohydrate. The filler may be present at various concentrations from about 30% w/w to about 78% w/w. For example, the filler may comprise about from about 30% w/w to about 78% w/w of lactose monohydrate and from about 0 to about 40% w/w of microcrystalline cellulose. For example, the filler could be lactose monohydrate in an amount of about 71% w/w.

In some embodiment the modified release tablet formulation comprises one or more glidants. The term “glidant” as used herein includes colloidal silicon dioxide, talc, etc. For example, the glidant could be colloidal silicon dioxide. The glidant may be present at various concentrations from about 0.1% w/w to about 2% w/w, for example from about 0.2% w/w to about 1% w/w, e.g. about 0.5% w/w.

In some embodiment the modified release tablet formulation further comprises one or more lubricants. The term “lubricant” as used herein includes magnesium stearate, sodium stearyl fumarate, talc, etc. For example, the lubricant may be magnesium stearate. The lubricant may be present at various concentrations from about 0.1% w/w to about 2% w/w, for example from about 0.5% w/w to about 1.5% w/w, e.g. about 1.0% w/w.

Suitably the % w/w of the components comprising the modified release tablets described herein (e.g. the compound of formula (I), the hydrophilic matrix former, the filler, glidant and/or lubricant) refer to the % w/w prior to adding the optional coating to the tablets. Thus as will be realised by the skilled person, in those embodiments where the modified release tablets are coated, the % w/w of each component in the coated tablet based on the total weight of the coated tablet will be lower than the % w/w based on the uncoated tablet cores due to the additional weight of the coating.

In some embodiments the modified release tablet formulation may comprise a film coating of the tablet cores. A suitable coating may be a PVA-based coating system. The term “coating system”, as used herein includes HPMC-based coating systems, PVA-based coating systems (polyvinyl alcohol), PVA-PEG based coating systems (polyethylene glycol) or ethylcellulose based functional barrier membrane coating systems. For example, the coating system could be the PVA-based coating system. For example, the coating system could be Opadry® II. For example the coating system could be present in an amount from about 3% to about 5% weight gain of the tablet formulation, for example a 4% weight gain.

In some embodiments, the particle size distribution of the compound in the tablet formulation may be D50≤25 μm, for example D50≤20 μm, D50≤10 μm, D50≤5 μm, or D50≤3 μm.

The amount of the compound of formula (I) in each tablet may range from about 1 mg to about 100 mg, or from about 5 mg to about 60 mg. The amount of the compound may for example range from 10 mg to 50 mg, from 20 mg to 45 mg, and from 30 mg to 40 mg. In some embodiments, the amount of the compound in each tablet may be from about 10 to about 30 mg. In some embodiments, the amount of the compound of formula (I) in each tablet is 1 mg, 2 mg, 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg or 100 mg.

In some embodiments, the compound of formula (I), or a pharmaceutically acceptable salt, solvate or hydrate thereof, is comprised within a granulated blend formulation. A granulated blend formulation may comprise:
the compound of formula (I), or a pharmaceutically acceptable salt, solvate or hydrate thereof; and
one or more of a pharmaceutically acceptable hydrophilic matrix former;
one or more pharmaceutically acceptable excipients selected from the group consisting fillers, binders, glidants and lubricants; and
a hard capsule shell material.

The hydrophilic matrix former could be hydroxypropyl methylcellulose (HPMC), hydroxypropylcellulose (HPC), or mixtures thereof. The hydrophilic matrix former could be present at various concentrations and combinations from about 10% w/w to about 20% w/w HPMC.

The fillers/binders could be selected from lactose monohydrate, lactose hydrous or microcrystalline cellulose, and mixtures thereof. The fillers/binders could be present at various concentrations from about 20% w/w to about 75% w/w of lactose monohydrate and from 0 to about 50% w/w of microcrystalline cellulose. The glidant could be colloidal silicon dioxide, which could be present at various concentrations from about 0.1% w/w to about 2% w/w.

The lubricant could be magnesium stearate, which could be present at various concentrations from about 0.1% w/w to about 2% w/w.

In some embodiments the compound of formula (I) is present in the granulated blend formulation in an amount of from about 1% w/w to about 40% w/w, for example about 1% w/w to about 30% w/w, from about 1% w/w to about 20% w/w, or from about 2% w/w to about 15% w/w.

The blend formulation could be dispensed in a hard capsule. Capsule shell material for hard capsules could be made of several materials such as gelatin (pig, bovine, fish etc), hydroxypropyl methylcellulose (HPMC), polyvinyl alcohol, starch and pullulan could be applied.

In some embodiments the granulated blend formulation is formulated as a unit dosage form (e.g. a capsule formulation). The amount of the compound of formula (I) in each unit dose form may range from about 1 mg to about 100 mg, or from about 5 mg to about 60 mg. The amount of the compound may for example range from 10 mg to 50 mg, from 20 mg to 45 mg, and from 30 mg to 40 mg. In some embodiments, the amount of the compound in unit dosage form comprising the granulated blend formulation may be from about 10 to about 30 mg. In some embodiments, the amount of the compound of formula (I) in each unit dosage form is 1 mg, 2 mg, 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg or 100 mg

A manufacturing process for the granulated blend formulation could consist of blending and sieving steps of the drug substance and excipients followed by granulation, e.g. roller compaction, and encapsulation.

In another aspect, the present invention relates to a method of treating HS, the method comprising administering to a subject in need thereof a modified release tablet formulation comprising the compound of formula (I), or a pharmaceutically acceptable salt, solvate or hydrate thereof.

### Formulations for Parenteral Administration

Formulations of the present invention suitable for parenteral (e.g. intravenous, intramuscular or subcutaneous) administration may be in the form of granules, powder or a concentrated liquid (e.g. a solution or suspension) which can be reconstituted or diluted prior to use by the addition of a liquid, such as an aqueous liquid (e.g. water or saline) to form an essentially clear, stable liquid comprising the formulation dissolved in solution. The reconstituted or diluted solution also forms part of the present invention.

### Treatment of Hidradenitis Suppurativa

The present invention provides a compound of formula (I), or a pharmaceutically acceptable salt, solvate or hydrate thereof, for use in the treatment of HS. Also provided is a method for treating HS, the method comprising administering to a subject in need thereof a therapeutically effective amount of a compound of formula (I), or a pharmaceutically acceptable salt, solvate or hydrate thereof.

HS is a chronic, inflammatory-mediated disease of the apocrine glands that presents clinically as bilateral painful nodules, abscesses, sinus tracts, and scarring in the axillae and the inguinocrural and anogential regions. The estimated worldwide prevalence of HS is 1-4%. Females are three times more likely to be affected than males, though males tend to have more severe disease (Napolitano et al. Clin Cosmet Investig Dermatol. 2017; 10:105-15). HS significantly impacts quality of life and mental health. The etiology of HS, though not completely understood, is multi-factorial and includes folliculopilosebaceous anatomical abnormalities, genetic mutations, immune dysregulation, endocrine influence, and an imbalance of surface and adnexal microbiota, in addition to modifiable factors, such as smoking and metabolic syndrome.

Several inflammatory mediators have been implicated in HS pathogenesis. Biopsies of HS lesions have revealed an abundance of neutrophilic granulocytes, T helper 1 T cells (Th1), T helper 17 T cells (Th17), macrophages, and dendritic cells. Cytokines involved include tumor necrosis factor-alpha (TNF-α), interferon-gamma (IFN-γ), and interleukins (IL-1β, IL10, IL-17A, and IL-23). In particular, TNF-α, a proinflammatory cytokine produced by innate and adaptive immune cells, has a critical role in several autoinflammatory diseases and the presence of high levels of TNF-α at the mRNA and protein levels in HS skin was shown in several studies (Mozeika et al., Acta Derm Venereol. 2013 May; 93(3):301-4). This is consistent with the clinical improvement that is reported with infliximab and adalimumab therapy. The presence of IL-1β, IL-23, and IL-17 in HS lesions have implicated the IL-1β-IL-23/TH17/IL-17 pathway in the pathogenesis of HS (Schlapbach et al., J Am Acad Dermatol. 2011 October; 65(4):790-798).

PDE4 is an enzyme that reduces levels of intracellular cyclic adenosine monophosphate (cAMP), a pro-inflammatory molecule that stimulates production of cytokines elevated in the serum and/or lesions of patients with HS, including TNF-α, IL-17, IL-23, and IL10. PDE4s are the predominant cAMP-degrading isozymes in most, if not all, immune and inflammatory cells, including T cells, B cells, eosinophils, neutrophils, dendritic cells, monocytes, and macrophages (Torphy, Am J Respir Crit Care Med 1998; 157:351-70). Three PDE4 subtypes, PDE4A, PDE4B and PDE4D, are expressed in these cells, while PDE4C is minimal or absent (Press et al., Prog Med Chem 2009; 47:37-74). It has been demonstrated that the pharmacological effects of PDE4 inhibitors on macrophage TNF-α production are mediated exclusively through inhibition of PDE4B (Jin et al., J Immunol 2005; 175: 1523-31; Jin et al., Proc Natl Acad Sci USA 2002; 99: 7628-33). PDE4D is a predominant subtype conducting “long-term” lymphocyte responses, such as IFN-γ and IL-5 release and proliferation (Peter et al., J Immunol 2007; 178: 4820-31). It has also been shown that ablation of PDE4D or PDE4B, but not PDE4A, has profound effects on neutrophil functions (Ariga et al., J Immunol 2004; 173:7531-8).

The side effects associated with PDE4 inhibitors are thought to be a result of their non-selectivity to all four PDE4 subtypes, and thus generation of new PDE4 inhibitors with subtype selectivity may provide clinical benefits by maintaining therapeutic efficacy while decreasing the side effects (Manning et al., Br J Pharmacol 1999; 128: 1393-8). This notion is supported by a series of studies in which PDE4 gene-targeted mice were used to define the function of individual PDE4 subtypes (Jin et al., Cyclic Nucleotide Phosphodiesterases in Health and Disease Boca Raton, FL: CRC Press, 2007:323-46).

In vitro pharmacology studies have demonstrated that the compound of formula (I) is a potent and selective PDE4 inhibitor, especially of the PDE4B and PDE4D subtypes. The compound has also been found to inhibit the secretion of tumour necrosis factor-alpha (TNF-α), interferon gamma (IFN-γ) and interleukin (IL)-1, -2 and -4. Furthermore, both oral and topical administration of the compound was shown to exhibit anti-inflammatory effects in a mouse model. These characteristics make the compound of formula (I) particularly suitable for the treatment of HS. Without being bound by theory, it is believed that the ability of the compound of formula (I) to specifically inhibit the PDE4 isoforms PDE4B and PDE4D, which are related to inflammation, may provide an improved therapeutic window compared with other PDE4 inhibitors, such as apremilast and roflumilast which are known to be broad unspecific inhibitors of PDE4. Subtype specificity may be of importance as recent research indicates that the order of importance for anti-inflammatory effects appears to be inhibition of PDE4B>PDE4D>PDE4A, with no or very limited beneficial effects from PDE4C inhibition. Furthermore, as PDE4B2 expression in the brain is low, this from a theoretical standpoint is a good target to reduce potential systemically driven adverse events. The compound of formula (I) thus offers a more optical approach to PDE4 inhibition which balances anti-inflammatory effects and tolerability, in particular reducing unwanted side effects associated with treatment, thereby providing an advantage over known PDE4 inhibitors.

In some embodiments, the compound of the invention is for use in treating a symptom of HS. For example, the compound may be for use in eliminating, or reducing the number, severity and/or spread of inflammatory nodules, abscesses, comedones and/or sinus tracts. In some embodiments the compound of the invention is for use in eliminating or reducing abscesses, nodules and/or draining fistulas caused by or associated with HS. In some embodiments the compound of the invention is for use in eliminating or reducing abscesses and/or nodules caused by or associated with HS.

In some embodiments, the compound is for use in reducing inflammation caused by or associated with HS. In some embodiments the compound is for use in reducing inflammation caused by or associated with HS, wherein the compound reduces one or more inflammatory biomarkers associated with HS in the subject. For example the compound of the invention may reduce one or more of: C-Reactive Protein (e.g. High-Sensitivity C-Reactive Protein (hs-CRP)); erythrocyte sedimentation rate; leukocyte count; or thrombocyte count relative to the baseline levels prior to treatment with the compound.

In some embodiments, the compound is for use in eliminating or reducing pruritis caused by or associated with HS.

In some embodiments, the compound is for use in eliminating or reducing swelling caused by or associated with HS. For example, the compound of formula (I) may reduce swelling of HS lesions.

In some embodiments, the compound is for use in eliminating or reducing scarring caused by or associated with HS.

In some embodiments the compound is for use in reducing the size of lesions associated with HS. For example, it may be that the compound is for use in reducing or eliminating lesions associated with HS.

In some embodiments, the compound is for use in reducing pain caused by or associated with HS. Pain may be assessed according to the Patient's Global Assessment of Skin Pain (0=no pain, 10=worst imaginable pain) 0-10 numerical rating scale (NRS) (Newton et al., J Patient Rep Outcomes. 2019; 3(1):42.). Suitably, NRS is reduced by at least 30%, compared to baseline. Other well-known pain scoring systems can be used to assess the reduction in pain associated with the HS. For example the reduction of pain could also be assessed using the Visual analog scale of pain (VAS pain), which also assesses pain on a visual scale of 0 (no pain) to 10 (worst imaginable pain).

Pain may also be assessed according to the McGill Pain questionnaire. The McGill Pain questionnaire can be used to evaluate the sensation, strength and change over time of experienced pain. It can monitor pain overtime or determine the effectiveness of intervention (Melzack, Pain: September 1975, Volume 1, Issue 3, p277-299).

In some embodiments the pain associated with HS is reduced by at least about 10%, 20%, 30%, 40%, 50%, 60%, 70%, 80% or 90% using a suitable pain scoring method (e.g. one of the scoring methods described herein) relative to the baseline pain level prior to treating the HS with the compound of formula (I).

In some embodiments, the Hidradenitis Suppurativa Quality of Life (HisQoL) total score of the patient treated with the compound is reduced during the treatment period. In some embodiments the HisQoL of the subject is reduced by at least about 10%, 20%, 30%, 40%, 50%, 60%, 70%, 80% or 90% relative to the baseline level prior to treating the HS with the compound. Suitably, the HisQoL of the patient is reduced by at least 50%, such as at least 75% or such as at least 90%.

The HiSQOL is based on the work of the Hidradenitis SuppuraTiva cORe outcomes set International Collaboration (HISTORIC). HiSQOL has been developed and validated systematically and is a 17-item questionnaire that contains HS specific items such as drainage and odor in addition to more general skin specific items. HiSQOL is a HS-specific questionnaire designed to evaluate HRQOL in clinical trials (Thorlacius et al., Skin Appendage Disord. 2019 June; 5(4):221-229; Kirby et al., Br_J Dermatol. 2020 August; 183(2):340-348). It has a recall-period of 7 days and consists of 17 items divided into three domains: Four symptom questions, five psychosocial questions, and eight activities adaptation questions. For each item a score between 0 and 4 is given, with a higher score representing a greater adverse impact on HRQOL. In some embodiments the HRQOL of the subject is reduced by at least about 10%, 20%, 30%, 40%, 50%, 60%, 70%, 80% or 90% relative to the baseline level prior to treating the HS with the compound.

In some embodiments, the Hidradenitis Suppurativa Clinical Response (HiSCR) of the patient treated with the compound is reduced during the treatment period. In some embodiments the HiSCR of the subject is reduced by at least about 10%, 20%, 30%, 40%, 50%, 60%, 70%, 80% or 90% relative to the baseline level prior to treating the HS with the compound. In some embodiments the HiSCR of the patient is reduced by at least 50%, such as at least 75% or such as at least 90%.

HiSCR is a treatment target based on correlation between the changes in lesion counts and PROM (pain and HRQoL). Hidradenitis Suppurativa Clinical Response (HiSCR; a 250% reduction from baseline in abscess and inflammatory nodule count and no increase in abscess or draining fistula counts) (Kimball et al., Ann Intern Med. 2012 Dec. 18; 157(12):846-55; Kimball et al., J Eur Acad Dermatol Venereol. 2016 June; 30(6):989-94). Subsequently modified for further differentiation: HiSCR75; a ≥275% reduction from baseline in abscess and inflammatory nodule count and no increase in abscess or draining fistula counts) and HiSCR-90; a ≥90% reduction from baseline in abscess and inflammatory nodule count and no increase in abscess or draining fistula counts)

In some embodiments, the Physician's Global Assessment of disease severity (HS-PGA) of the patient treated with the compound is reduced during the treatment period. Suitably, the HS-PGA of the patient during or after treatment is reduced to a score of 0 or 1.

HS-PGA ranges from clear to very severe (Kimball et al., Ann Intern Med. 2012 Dec. 18; 157(12):846-55). It is used in clinical trials to measure clinical improvement in inflammatory nodules, abscesses, and draining fistulae. The six stages are;
Clear: No inflammatory or non-inflammatory nodules.
Minimal: Only the presence of non-inflammatory nodules.
Mild: Less than 5 inflammatory nodules or 1 abscess or draining fistula and no inflammatory nodules.
Moderate: Less than 5 inflammatory nodules, or 1 abscess or draining fistula and 1 or more inflammatory nodules, or 2-5 abscesses or draining fistulas and less than 10 inflammatory nodules.
Severe: 2-5 abscesses or draining fistulas and 10 or more inflammatory nodules.
Very severe: More than 5 abscesses or draining.

In some embodiments the compound is for use in reducing the severity of the HS. For example it may be that the compound reduces the severity by one or more (e.g. 1, 2 or 3) HS-PGA levels. Thus it may be that the compound reduces the severity of the HS from very severe to severe, moderate or mild HS.

In some embodiments, the compound reduces the amount of C-Reactive Protein (e.g. High-Sensitivity C-Reactive Protein (hs-CRP)) in the patient treated with the compound. Suitably, the amount of C-Reactive Protein (e.g. hs-CRP) in the patient is reduced by at least about 10%, 20%, 30%, 40%, 50%, 60%, 70%, 80% or 90% relative to the baseline level prior to treating the HS with the compound. For example the amount of C-Reactive Protein (e.g. hs-CRP) is reduced by at least 50%, such as at least 75% or such as at least 90%.

In some embodiments, the Dermatology Life Quality Index (DLQI) of the patient treated with the compound is reduced during the treatment period. In some embodiments the DLQI of the subject is reduced by at least about 10%, 20%, 30%, 40%, 50%, 60%, 70%, 80% or 90% relative to the baseline level prior to treating the HS with the compound. In some embodiments, the DLQI of the patient is reduced by at least 50%, such as at least 75% or such as at least 90%.

DLQI is a questionnaire of 10 questions concerning the patients' perception of the impact of skin diseases on different aspects of their health-related quality of life over the last week. Each question is scored on a four-point scale (0-3) resulting in a range of 0-30 points (0=Disease has no impact on quality of life, 30=Disease has maximum impact on quality of life). A validated scale first introduced by Finlay and Khan, Clin. Exp. Dermatol., 19 (1994), pp. 210-216).

In some embodiments, the Work Productivity and Activity Questionnaire (WPAI) impairment percentage of the patient treated with the compound is reduced during the treatment period. Suitably, the impairment score of the patient is reduced by at least 50%, such as at least 75% or such as at least 90%.

WAPI is a questionnaire describing work impairment due to a specific disease. Outcomes are expressed as impairment percentages, with higher numbers indicating greater impairment and less productivity (Reilly et al., Pharmacoeconomics. 1993 November; 4(5):353-65).

In some embodiments, the Anxiety and Depression (HADS) score of the patient treated with the compound is reduced during the treatment period. Suitably, the HADS score of the patient is reduced by at least 50%, such as at least 75% or such as at least 90%.

HADS is a questionnaire comprising seven questions for anxiety and seven questions for depression. Each question is connected to four answers retrieving 0-3 points. For each condition, 0-7 points corresponds to normal case; 8-10 to borderline abnormal; and 11-21 to abnormal case (Zigmond and Snaith, Acta Psychiatrica Scandinavica (1983), 67(6): 361-370).

In some embodiments, the European quality of life-5 Dimensions (EQ-5D) score of the patient treated with the compound is increased during the treatment period. Suitably, the EQ-5D score of the patient is increased by at least 50%, such as at least 75% or such as at least 90%.

EQ-5D is a standardized instrument for measuring generic health status in terms of five dimensions: mobility, self-care, usual activities, pain/discomfort, and anxiety/depression. Each dimension receives a value of 1-5 leaving 55 55) different health states. The score is combined with an overall patient rating of health from 0-100 where 0 is worst imaginable health and 100 is best imaginable health. The scale was further developed from the original EQ-5D by Herdman et al., Qual Life Res. 2011 December; 20(10):1727-36.

In some embodiments, the Multidimensional Fatigue Inventory 20 (MFI-20) response of the patient treated with the compound is improved during the treatment period.

MFI-20 was invented by Smets et al., J Psychosom Res 1995; 39: 315-25. It consists of 20 items describing five subscales of fatigue: General Fatigue (GF), Physical Fatigue (PF), Reduced Motivation (RM), Reduced Activity (RA), and Mental Fatigue (MF). For each of the items the respondent must specify the extent to which the particular statements relate to him/her on a five-point scale, ranging from Yes, that is true to No, that is not true.

Subjects with HS are prone to flares in the disease in which the severity of the HS increases, for example wherein the abscess and nodule count increases by at least 25% compared to baseline. In some embodiments the compound of formula (I) is for use in preventing or reducing HS flares in the subject. In some embodiments the compound of formula (I) is for use in reducing the severity of HS flares in the subject. In some embodiments the compound of formula (I) is for use in reducing the frequency of HS flares in the subject. In some embodiments the compound of formula (I) is for use in reducing the frequency and severity of HS flares in the subject.

In a further aspect, the invention provides a method of treating a subject with HS, the method comprising administering to the subject a compound of formula (I), or a pharmaceutically acceptable salt, solvate or hydrate thereof. The method may comprise reducing inflammation associated with HS.

In another aspect, the invention provides the use of a compound of formula (I), or a pharmaceutically acceptable salt, solvate or hydrate thereof, for the manufacture of a medicament for use in the treatment of HS.

The HS may be mild, moderate or severe. The severity (also referred to as the disease state or progression) is defined according to the International Hidradenitis Suppurativa Severity Score System (IHS4), which is a validated international clinimetric scale (Zouboulis et al., Br J Dermatol. 2017; 177(5):1401-1409). The score is based on a count of inflamed lesions. The resulting IHS4 score is arrived at by the number of nodules (multiplied by 1) plus the number of abscesses (multiplied by 2) plus the number of draining tunnels (multiplied by 4). A total score of 3 or less signifies mild, 4-10 signifies moderate and 11 or higher signifies severe disease.

In some embodiments the subject has mild HS. In some embodiments the subject has moderate HS. In some embodiments the subject has severe HS.

In some embodiments, the compound is for use in reducing the severity of the HS. It may be that the treatment is for reducing the severity from severe to moderate HS or from moderate to mild HS. In some embodiments. In some embodiments, the treatment is for reducing the IHS4 score of the HS.

In some embodiments, the compound is for use in preventing the progression of HS from mild to moderate HS, or from moderate to severe HS.

The subject may also be suffering from a comorbidity selected from obesity, metabolic syndrome, inflammatory bowel disease, spondyloarthropathy, or any combination thereof.

It may be that the subject has not been previously treated with antibody therapy. Additionally or alternatively, it may be that the subject has not been previously treated with a TNF-α inhibitor. It may be that the subject has not been previously treated with a biological therapy for HS.

In a further embodiment, the subject has been previously been treated with a biological therapy for HS. It may be that the subject is non-responsive or refractory to treatment with a biological therapy for HS.

In another embodiment the subject is non-responsive or refractory to one or more HS therapy other than the compound of formula (I). In some embodiments the subject is non-responsive or refractory to one or more HS therapy selected from an antibiotic (e.g. dapsone, doxycycline, clindamycin, rifampin or a carbapenem (e.g. ertapenem)) and a biological therapy for HS (e.g. any of the biological therapies for HS disclosed herein).

Reference herein to a “biological therapy for HS” includes anti-TNF-α biologics (e.g. adalimumab, certolizumab infliximab, etanercept, or golimumab); anti-IL-17 biologics (e.g. bimekizumab, brodalumab, CJM112, ixekizumab or secukinumab); anti-IL-12/23 biologics (e.g. ustekinumab), anti-IL-23 biologics (e.g. guselkumab, risankizumab, or tildrakizumab); an anti-IL-1 biologic (e.g. anakinra, bermkimab or canakinumab); an anti CD (e.g. iscalimab); or an anti-IL-36 biologic (e.g. spesolimab or ismidolimab); anti CXCR1/CXCR2 biologics (e.g. LY 3041658), or a Complement C5a inhibitor, or any combination thereof. In some embodiments the biological therapy for HS is an anti-TNF-α biologic (e.g. adalimumab or infliximab). In some embodiments the biological therapy for HS is adalimumab.

The subject may be a human or an animal. In some embodiments the subject is a human. The subject may be from 10 to 50 years, from 15 to 40 years or from 20 to 30 years of age. In some embodiments the subject is, or has previously been, a smoker. In some embodiments the subject has a BMI of at least 30, at least 40 or above.

### Combination Therapies

The compound of the invention, or a formulation or composition comprising the compound, may be used alone to provide a therapeutic effect. The compound of the invention, or a formulation or composition comprising the compound, may also be used in combination with a further therapy.

In some embodiments the further therapy is selected from an anti-androgenic agent, a hormone, an antibiotic (e.g. dapsone, doxycycline, clindamycin, rifampin or a carbapenem (e.g. ertapenem)), a retinoid, an anti-inflammatory agent (including steroids, non-steroidal anti-inflammatory agents and colchicine), an analgesic, an immunosuppressive agent, an antibody, surgery, metformin, a nutritional supplement (e.g. zinc gluconate), a biological therapy for HS (e.g. a TNF-α inhibitor (e.g. adalimumab or infliximab), an IL-1 inhibitor (e.g. anakinra), an anti-IL-17 drug (e.g. secukinumab), an anti-IL-23 drug (e.g. risankizumab), or an anti-IL-12/23 drug (e.g. ustekinumab)), a complement C5a inhibitor (e.g. avacopan), a Janus Kinase (JAK) inhibitor (e.g. tofacitinib, INCB054707 or upadacitinib), a leukotriene A4 hydrolase inhibitor (e.g. LYS 006), an IRAK4 degrader (e.g. KT-474), a IRAK4 inhibitor (e.g. PF-06650833), a tyrosine kinase 2 (TYK2) inhibitor (e.g. PF-06826647) or a TYK2/JAK1 inhibitor (e.g. PF-06700841), or any combination thereof. Thus it may be that the further therapy is selected from an anti-androgenic agent, a hormone, an antibiotic (e.g. dapsone, doxycycline, clindamycin, rifampin or a carbapenem (e.g. ertapenem)), a retinoid, an anti-inflammatory agent (including steroids, non-steroidal anti-inflammatory agents and colchicine), an analgesic, an immunosuppressive agent, an antibody, surgery, metformin, a nutritional supplement (e.g. zinc gluconate), a TNF-a inhibitor (e.g. adalimumab), and IL-1 inhibitor (e.g. anakinra), an anti-IL-17 drug (e.g. secukinumab), and anti-IL-23 drug (e.g. risankizumab), and anti-IL-12/23 drug (e.g. ustekinumab), a Janus Kinase (JAK) inhibitor (e.g. tofacitinib), or any combination thereof.

Such combination treatment may be achieved by way of the simultaneous, sequential or separate dosing of the individual components of the treatment. Such combination products employ the formulation of this invention within a therapeutically effective dosage range described hereinbefore and the other pharmaceutically-active agent within its approved dosage range.

Herein, where the term “combination” is used it is to be understood that this refers to simultaneous, separate or sequential administration. In one aspect of the invention “combination” refers to simultaneous administration. In another aspect of the invention “combination” refers to separate administration. In a further aspect of the invention “combination” refers to sequential administration. Where the administration is sequential or separate, the delay in administering the second component should not be such as to lose the beneficial effect of the combination.

In some embodiments in which a combination treatment is used, the amount of the compound of the invention and the amount of the other pharmaceutically active agent(s) are, when combined, therapeutically effective to treat a targeted disorder in the patient. In this context, the combined amounts are “therapeutically effective amount” if they are, when combined, sufficient to reduce or completely alleviate symptoms or other detrimental effects of the disorder; cure the disorder; reverse, completely stop, or slow the progress of the disorder; or reduce the risk of the disorder getting worse. Typically, such amounts may be determined by one skilled in the art by, for example, starting with the dosage range described in this specification for the compound of formula (I), or a pharmaceutically acceptable salt, solvate or hydrate thereof present in the formulation of the invention and an approved or otherwise published dosage range(s) of the other pharmaceutically active agent(s).

### Dosages and Dosage Regimens

The dosage and dosing regimen of the formulation of the invention will depend upon a number of factors that may readily be determined by a physician, for example the severity of the disease or condition, the responsiveness to initial treatment, the mode of administration and the particular disease or condition being treated. Examples of suitable doses, dosing volumes and frequencies are set out in the brief summary of the disclosure above.

Suitable modes of administration include oral, intranasal, parenteral (e.g. intravenous, intramuscular, intra-arterial, subcutaneous or intradermal), topical, inhalation (intraorally or intranasally), or a combination thereof. One or more doses may be delivered to the subject via multiple modes of administration.

The total daily dose of the compound of formula (I) administered to the subject may comprise one or more unit doses. The total daily dose may be no more than 120 mg, no more than 100 mg, no more than 80 mg, no more than 60 mg, or no more than 40 mg of the compound of formula (I). In some embodiments, the treatment comprises administering a total daily dose of at least 20 mg, at least 30 mg, at least 40 mg, at least 50 mg, at least 60 mg, at least 80 mg, or at least 100 mg of the compound of formula (I).

In some embodiments the total daily dose administered is from about 10 to about 120 mg, from about 20 to about 110 mg, from about 30 to about 100 mg, from about 40 to about 90 mg, from about 50 to about 80 mg, or from about 60 to about 70 mg of the compound of formula (I) or salt, solvate or hydrate thereof.

In some embodiments, the treatment comprises administering a unit dose of from about 1 mg to about 100 mg, from about 5 mg to about 70 mg, from about 8 mg to about 65 mg, from about 10 mg to about 60 mg, from about 15 mg to about 50 mg, from about 20 mg to about 45 mg, or from about 30 to about 40 mg. In some embodiments, the treatment comprises administering a unit dose of from about 1 mg to about 50 mg, from about 5 mg to about 40 mg, or from about 10 mg to about 30 mg. In some embodiments, the treatment comprises administering a unit dose of from about 5 mg. In some embodiments, the treatment comprises administering a unit dose of from about 10 mg. In some embodiments, the treatment comprises administering a unit dose of from about 20 mg. In some embodiments, the treatment comprises administering a unit dose of from about 30 mg. In some embodiments, the treatment comprises administering a unit dose of from about 40 mg. In some embodiments, the treatment comprises administering a unit dose of from about 50 mg. In some embodiments, the treatment comprises administering a unit dose of from about 60 mg. In some embodiments, the treatment comprises administering a unit dose of from about 70 mg. In some embodiments, the treatment comprises administering a unit dose of from about 80 mg. In some embodiments, the treatment comprises administering a unit dose of from about 100 mg.

The treatment may be administered from one to four times daily, for example from two to three times daily. In some embodiments the treatment is administered once daily. In some embodiments the treatment is administered twice daily.

Accordingly, in some embodiments the compound of formula (I) is administered in a dose of 5 mg once per day. in some embodiments the compound of formula (I) is administered in a dose of 10 mg once per day. In some embodiments the compound of formula (I) is administered in a dose of 20 mg once per day. In some embodiments the compound of formula (I) is administered in a dose of 30 mg once per day. In some embodiments the compound of formula (I) is administered in a dose of 40 mg once per day. In some embodiments the compound of formula (I) is administered in a dose of 50 mg once per day. In some embodiments the compound of formula (I) is administered in a dose of 60 mg once per day. In some embodiments the compound of formula (I) is administered in a dose of 70 mg once per day. In some embodiments the compound of formula (I) is administered in a dose of 80 mg once per day. In some embodiments the compound of formula (I) is administered in a dose of 90 mg once per day. In some embodiments the compound of formula (I) is administered in a dose of 100 mg once per day.

In some embodiments the compound of formula (I) is administered in a dose of 5 mg twice per day In some embodiments the compound of formula (I) is administered in a dose of 10 mg twice per day. In some embodiments the compound of formula (I) is administered in a dose of 20 mg twice per day. in some embodiments the compound of formula (I) is administered in a dose of 30 mg twice per day. In some embodiments the compound of formula (I) is administered in a dose of 40 mg twice per day. In some embodiments the compound of formula (I) is administered in a dose of 50 mg twice per day.

The compound may be administered to the subject over a number of consecutive days or weeks. In some embodiments the compound is administered one or more times daily over a period of at least 10 days, at least 2 weeks, at least 3 weeks, at least 4 weeks, at least 6 weeks, at least 8 weeks, at least 10 weeks, at least 12 weeks, at least 16 weeks, at least 20 weeks, or at least 6 months. In some embodiments, the treatment is administered for no more than 12 months, no more than 10 months, no more than 9 months, no more than 8 months, no more than 6 months, no more than 24 weeks, no more than 20 weeks, no more than 16 weeks, no more than 12 weeks or no more than 10 weeks. For example, the compound may be administered for a period of from 10 days to 20 weeks, from 14 days to 16 weeks, from 21 days to 14 weeks, from 4 to 12 weeks, from 5 to 10 weeks or from 6 to 8 weeks. In some embodiments, the compound is administered to the subject twice daily for up to 4, 6, 10, 16 or 20 weeks. Alternatively, it may be that the treatment is administered for a longer period of time, for example, for maintenance e.g. in mild cases of HS. Thus, in some embodiments the treatment is administered for a period of time of from 6 months to 5 years, from 12 months to 4 years, from 15 months to 3 years, or from 18 to 24 months. It will be appreciated that the dosing period will be determined by the type and severity of the disease being treated. It may be that treatment is continued until the number of inflammatory nodules, abscesses, comedones and/or sinus tracts on the subject has been substantially reduced or eliminated. It may be that treatment is continued until the subject's IHS4 score has been reduced by at least 1, at least 2, at least 3 or more integers. It may be that treatment is continued until the severity of the HS has been reduced from very severe to severe, from very severe to moderate, from severe to moderate or from moderate to mild.

In one embodiment, the dose of the compound of administered is increased over the first two weeks of treatment. Suitably, the dose is increased from 10 mg twice daily to 30 mg twice daily over the period of two weeks. In some embodiments the compound is administered in an initial total daily dose of about 5 mg to 20 mg and the total daily dose is increased over a period of 1 or 2 weeks. For example the initial daily dose may be 5 mg to 20 mg and this is increased to a daily dose of 50 to 100 mg over a period of 1 or 2 weeks. In some embodiments in an initial total daily dose of about 5 mg to 20 mg and the total daily dose is increased over a period of 1 or 2 weeks to a total daily dose of about 60 to 80 mg per day. In these embodiments the daily dose may be administered as a single daily dose of the compound. However, suitably the total daily dose is administered as a divided dose administered at regular intervals. For example the total daily dose may be administered as substantially equal divided doses 2 to 4 times per day. Suitably the total daily dose is administered as a substantially equal divided dose 2 times per day. In some embodiments the compound is administered for the first two weeks of treatment substantially as described in the “Dose Titration Scheme” in Table 15 in the Examples herein.

It will be appreciated that the dose of the formulation and/or the dosage regime may be selected by the skilled person depending on a number of factors such as, but not limited to, the severity of the disease, the age of the subject and/or the presence of any underlying conditions.

### EXAMPLES

The invention is further illustrated by the following examples.

### Example 1: Compositions

Embodiments of compositions for oral administration comprising the compound of formula (I) are shown in Table 1:

TABLE 1
Compositions comprising compound (I) for oral administration

| | | | | 4: blend, | 5: gastro- |
|---|---|---|---|---|---|
| | 1: hard | 2: modified | 3: soft | hard | resistant |
| | capsule | release tablet | capsule | capsule | capsule |
| core | compound (I) | compound (I) | compound (I) | compound (I) | compound (I) |
| ingredients | | | | | |
| | | Lactose | Triglyceride, | Microcrystalline | Microcrystalline |
| | | monohydrate | medium chain | cellulose | cellulose |
| | | Hypromellose | Lecithin | Lactose | Lactose |
| | | | | monohydrate | monohydrate |
| | | Silica, | Hard fat | Hypromellose | Coscarmellose |
| | | colloidal | | | sodium |
| | | anhydrous | | | |
| | | Magnesium | Silica, | Silica, | Silica, |
| | | stearate | colloidal | colloidal | colloidal |
| | | | anhydrous | anhydrous | anhydrous |
| | | | | Magnesium | Magnesium |
| | | | | stearate | stearate |
| shell/coating | Gelatin | Macrogol | Gelatin | Gelatin | Hypromellose |
| | Ferric | PVA | Glycerol | Ferric | Hypromellose |
| | oxide red | | | oxide red | acetate succinate |
| | Titanium | Titanium | Ferric | Titanium | Titanium |
| | dioxide | dioxide | oxide red | dioxide | dioxide |
| | | Talc | Water, | | |
| | | | purified | | |
| | | Ferric oxide | | | |
| | | yellow | | | |

### Example 2: Inhibition of PDE Enzymes

PDE4 enzymatic activity was measured in a scintillation proximity assay using purified human recombinant PDE4D protein. The results are shown in Table 2:

TABLE 2
Inhibition of PDE4

EC₅₀ values
Compound of formula (I)

| | | |
|---|---|---|
| PDE4D SPA* | 14.7 nM | 7.5 ng/mL |

*SPA = scintillation proximity assay

Inhibition of PDE4 subtypes by the compound of formula (I) was tested using IMAP technology, which is based on the high affinity binding of phosphate by immobilized metal coordination complexes on nanoparticles. The binding reagent complexes with phosphate groups on nucleotide monophosphate generated from cyclic nucleotides (cAMP/cGMP) through phosphodiesterases. With fluorescence polarisation detection, binding causes a change in the rate of the molecular motion of the phosphate bearing molecule, and results in an increase in the fluorescence polarization value observed for the fluorescent label attached to the substrate.

Stocks of the compound of formula (I) were prepared in 100% DMSO. All assays were performed in 3% (final) DMSO for the IMAP assay. The compound was tested in duplicate against the PDE isoform panel at a range of concentrations (10⁻¹⁰, 10⁻⁹, 10⁻⁸, 10⁻⁷ M). The results of the assay are shown in Table 3.

TABLE 3
Inhibition of PDE4 subtypes

| PDE | Concentration of | |
|---|---|---|
| isoform | compound (I) (M) | Inhibition (%) |
| PDE4A4 | 10⁻⁷ | 47 |
| | 10⁻⁸ | 24 |
| | 10⁻⁹ | 0 |
| | 10⁻¹⁰ | 0 |
| PDE4B2 | 10⁻⁷ | 61 |
| | 10⁻⁸ | 41 |
| | 10⁻⁹ | 18 |
| | 10⁻¹⁰ | 12 |
| PDE4C2 | 10⁻⁷ | 15 |
| | 10⁻⁸ | 2 |
| | 10⁻⁹ | 0 |
| | 10⁻¹⁰ | 9 |
| PDE4D3 | 10⁻⁷ | 74 |
| | 10⁻⁸ | 24 |
| | 10⁻⁹ | 0 |
| | 10⁻¹⁰ | 0 |

The compound of formula (I) inhibited PDE4B and PDE4D most potently, but also PDE4A to a lesser extent. PDE4C was not significantly inhibited at the highest dose tested, 100 nM. The compound of formula (I) inhibited PDE4D splice variant 3 with somewhat higher EC₅₀ value than the inhibition of PDE4D shown in Table 3. This difference is most likely due to different assay techniques used. Without being bound by theory, it is thought that the selectivity of the compound of formula (I) for certain PDE4 subtypes may be beneficial, for example by reducing side effects.

### Inhibition of PDE Enzymes in a Radiometric PDE Assay

The compound of formula (I) (orismilast) and the PDE inhibitors apremilast and roflumilast were assessed in a radiometric PDE assay using a panel of human PDE4 isoforms (PDE4A1, PDE4A4, PDE4A10, PDE4B1, PDE4B2, PDE4B3, PDE4C2, PDE4D1,PDE4D2, PDE4D3, PDE4D4, PDE4D5 and PDE4D7).

Enzyme Preparations: Partially purified human recombinant phosphodiesterases (PDEs) were obtained by cloning cDNA and expressing in S. frugiperda insect cells using a baculovirus expression system. Cells are harvested from culture by centrifuging at 400×g for 5 minutes. Cell pellet is resuspended in 20 ml of RIPA Buffer (150 mM NaCl, 10 mM Tris, 0.1% NP-40, pH8.3), 4 ml/200 ml of culture, plus protease inhibitors (100 μl/10 ml) and incubated on ice for 10-20 minutes then spun at 3500×g for 10 minutes at 4° C. Supernatant is kept and the pellet thrown away.

Radiometric PDE Assay: The assay consists of a two step procedure. Tritium-labelled cyclic AMP is hydrolysed to 5′-AMP o by PDE. The 5′-AMP is then further hydrolysed to adenosine by nucleotidase in snake venom. An anion-exchange resin binds all charged nucleotides and leaves [³H]adenosine as the only labelled compound to be counted by liquid scintillation. The radiometric assay method is a modification of the two-step method described in Thompson et al., Biochemistry 10; 311-316; 1971, adapted for 96 well plate format.

50 μl of diluted PDE enzyme was incubated with 50 μl of [³H]-cAMP (specific activity 25 Ci/mmol) and 11 μl of 50% DMSO (or compound dilution) for 20 minutes at 30° C. The enzyme was diluted in 20 mM Tris HCl pH7.4 (×2.2 final concentration) and [3H]-cAMP was diluted in 10 mM MgCl2, 40 mM Tris HCl pH 7.4 (×2.2 final concentration). Reactions were carried out in Greiner 96 deep well 1 ml master-block. The plates were centrifuged for 9 seconds before the 20 minutes incubation. The reaction was terminated by denaturing the PDE enzyme (at 70° C. for 2 minutes, plates were left to cool on ice for 10 minutes) after which 25 μl snake venom nucleotidase (225 μg/ml final concentration) was added and incubated for 10 minutes (at 30° C.), plates were centrifuged for 9 seconds before incubation. After incubation 200 μl Dowex resin (1×8, 200-400 mesh) was added and the plate shaken for 20 minutes then centrifuged for 3 minutes at 1000×g. 50 μl of supernatant was removed and added to 200 μl of MicroScint-20 in white plates (Greiner 96well Optiplate) and shaken for 30 minutes before reading on Perkin Elmer TopCount Scintillation Counter. IC₅₀ values were calculated using GraphPad Prism software (version 8).

### Results

The Ic₅₀ values (nM) are shown in Table 4:

TABLE 4

| | IC₅₀ (nM) | | |
|---|---|---|---|
| | | Orismilast | |
| PDE | Apremilast | (Compound (I)) | Roflumilast |
| PDE4A1 | 78 | 16 | 0.5 |
| PDE4A4 | 42 | 11 | 0.3 |
| PDE4A10 | 140 | 52 | 1.5 |
| PDE4B1 | 61 | 16 | 0.6 |
| PDE4B2 | 97 | 6 | 0.4 |
| PDE4B3 | 117 | 3 | 0.3 |
| PDE4C2 | 244 | 104 | 2.3 |
| PDE4D1 | 44 | 9 | 0.5 |
| PDE4D2 | 54 | 9 | 0.2* |
| PDE4D3 | 54 | 8 | 0.2 |
| PDE4D4 | 41 | 8 | 0.3 |
| PDE4D5 | 61 | 3 | 0.2 |
| PDE4D7 | 50 | 3 | 0.1 |

*value generated by extrapolation of data

### Example 3: Cell-Based TNF-α Release Assay

TNF-α, Serumfree, hPBMC, LPS

Cultures of human PBMC isolated from buffy coats were stimulated with lipopolysaccharide to activate the monocytes and to release TNF-α. PBMC were isolated from fresh buffy coats and frozen for later use. On the assay day, cells were thawed, washed in serum free medium (RPMI1640 with 25 mM HEPES, 1% pen/strep, 200 mM L glutamine and 0.5% human serum albumin) and living cells were counted. Test compounds were diluted in DMSO and further diluted in serum-free medium and pipetted into wells of 384 well tissue culture plates. LPS was added to the cells which shortly thereafter were added to the wells with titrated test compounds and incubated for 18 hours at 37° C. The next day, the level of TNF-α in the culture supernatant was quantitated by homogenous time-resolved fluorescence (HTRF) where FRET was measured at 665 nm and normalized to the fluorescence of europium cryptate at 620 nm. The effect (inhibition of TNF-α release) of a test compound was calculated using equation 1:

$$\% Effect = 100 * ( Sx - So ) / ( Sc - So ) .$$

Fluorescence intensities, measured at 665 nm and 620 nm, were used to calculate the 665/620 ratio as an estimate of the level of secreted TNF-α. Sx denotes the 665/620 ratio associated with the test compound. So and Sc denote the 665/620 ratios associated with 0.1% DMSO and 1E-05 M compound (II), respectively.

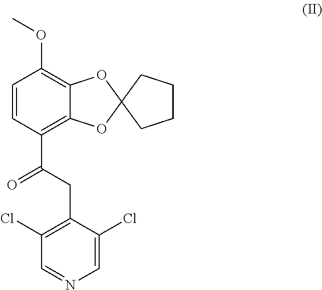 [SMILES: COc1ccc(C(=O)Cc2c(Cl)cncc2Cl)c2c1OC1(CCCC1)O2]

Viability, Serum-Free, hPBMC, LPS

The viability of the cells from the TNF-α assay is measured in the wells using the ATP vialight kit (Perkin Elmer, ATP-lite M, cat. no 6016949). The assay detects the amount of ATP using a bioluminescent method that utilizes an enzyme, luciferase, which catalyses the formation of light from ATP and luciferin according to the following reaction:

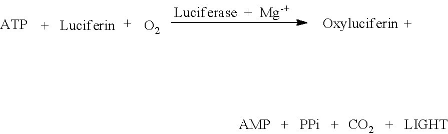

The emitted light intensity is linearly related to the ATP concentration and is measured using a luminometer. The effect (inhibition of viability) of a test compound was calculated using equation 1:

$$\% Effect = 100 * ( Sx - So ) / ( Sc - So )$$

where
So=Emitted light intensity reflecting the ATP concentration in the presence of 0.1% DMSO,
Sc=Emitted light intensity reflecting the ATP concentration in the presence of 10 μM Actinomycin in 0.1% DMSO and
Sx=Emitted light intensity reflecting the ATP concentration in the presence of test compound in 0.1% DMSO.

### Data Analysis

Molar concentrations of an inhibitor that produces 50% of the maximum possible “inhibitory” response (EC50 value), which is associated with the positive ctrl, is calculated according to equation General sigmoidal curve with Hill slope, a to d. This model describes a sigmoidal curve with an adjustable baseline, a. The equation can be used to fit curves where response is either increasing or decreasing with respect to the independent variable, X.

$$\% Effect = \frac{{E}_{Max} - {E}_{Min}}{1 + {( \frac{C}{Rel {EC}_{50}} )}^{{n}_{H}}} + {E}_{Min}$$

where
C=concentration of test compound
% Effect=normalized response calculated as described in Equation 1
Emin=min response as compound concentration approaches 0
Emax=max response as concentration of tested compound is increasing
EC50=Concentration of tested compound giving a response in the middle of Emin and Emax
nH=Hill coefficient or curve slope.

### Results

The compound of formula (I) was found to be potent inhibitor of monocyte-derived (lipopolysaccharide-induced) TNF-α, as shown in Table 5:

TABLE 5
Effect of compound (I) on TNF-α release
from human peripheral blood mononuclear cells

| | TNF-a assay | | Viability | |
|---|---|---|---|---|
| | EC50, nM | Emax, % | EC50, nM | Emax, % |
| Compound (I) | 11.4 nM | 102 | >9990 | 7% |

### Example 4: Effect of PDE4 Inhibitors on TNF-α Secretion from Human Whole Blood

The effect of the compound of formula (I) and two other PDE4 inhibitors, apremilast and roflumilast N-oxide, were studied in a whole blood TNF-α secretion assay. The JAK inhibitor, Tofacitinib was included as a non-PDE4 inhibitor reference compound. The tested PDE4 inhibitors showed a similar Emax but different EC₅₀ values in the various donors and assays. The compound of formula (I) was shown to be equipotent to—or slightly more potent than—roflumilast-N-oxide and an average of 23 times more potent than apremilast on a molar basis, in both LPS and SEB-induced TNF-α secretion from human whole blood.

### Materials and Methods

In the first experiment, several setups were tested. Freshly drawn human peripheral blood stabilized with heparin was diluted either with X-vivo-15 medium or with RPMI1640, and stimulated with different concentrations of lipopolysaccharide (LPS) or Staphylococcus enterotoxin B (SEB) for different time points. The compound of formula (I) was tested at 3 different concentrations in all the different assay setups.

In the second experiment all four compounds were tested in full titration in two donors. Xvivo 15 was used for dilution of the blood and the following four assay versions were run:
LPS 1 μg/mL for 24 hours
LPS 1 μg/mL for 48 hours
SEB 1 μg/mL for 24 hours
SEB 1 μg/mL for 48 hours

Test compounds were diluted in DMSO and added in duplicates to 384 well plates to give a final concentration of 0.1% DMSO in the wells. The blood was diluted with X-vivo 15 medium and added to the wells to give a volume of 80 μL per well and a final dilution of 50% blood/50% medium. The plates were placed in a water bath box in an incubator for 24 or 48 hours. TNF-α in the supernatants was measured by alpha-LISA kits (Perkin Elmer cat. No AL208F).

### Results

Table 6 shows the EC₅₀ values and Emax values obtained with the four compounds in the 4 different assay versions. The Emax was defined as the level of TNF-α in un-stimulated wells.

The Emax for the three PDE4 inhibitors was very similar within one donor and one assay version, but the EC₅₀ value was very different reflecting different potencies of the compounds. The JAK inhibitor, Tofacitinib, induced increased TNF-α secretion in the LPS stimulated assay version but inhibited the SEB stimulated secretion.

TABLE 6
EC₅₀ and Emax values of the test compounds

| | | 24 hours | | | | 48 hours | | | |
|---|---|---|---|---|---|---|---|---|---|
| | | donor 1 | | donor 2 | | donor 1 | | donor 2 | |
| | | EC₅₀ | Emax | EC₅₀ | Emax | EC₅₀ | Emax | EC₅₀ | Emax |
| LPS | Roflumilast | 2.34E−08 | 72 | 2.40E−08 | 79 | 3.86E−08 | 75 | 3.50E−08 | 78 |
| | N-oxide | | | | | | | | |
| | Compound | 5.34E−09 | 76 | 1.84E−08 | 79 | 1.21E−08 | 71 | 1.98E−08 | 80 |
| | (I) | | | | | | | | |
| | Apremilast | 2.10E−07 | 74 | 2.83E−07 | 79 | 2.77E−07 | 77 | 3.64E−07 | 78 |
| | Tofacitinib | Stimulation | | | | Stimulation | | | |
| SEB | Roflumilast | 8.69E−09 | 97 | 3.56E−08 | 66 | 4.77E−09 | 97 | 3.34E−08 | 80 |
| | N-oxide | | | | | | | | |
| | Compound | 4.93E−09 | 97 | 2.49E−08 | 66 | 4.66E−09 | 98 | 4.78E−08 | 83 |
| | (I) | | | | | | | | |
| | Apremilast | 1.39E−07 | 98 | 6.20E−07 | 65 | 1.55E−07 | 97 | 5.73E−07 | 80 |
| | Tofacitinib | 1.68E−07 | 98 | 8.30E−06 | 100* | 1.82E−07 | 95 | 1.45E−06 | 102 |

*Locked value, no top plateau

Table 7 shows the mean EC₅₀ value upon pooling the results from all eight whole blood TNF-α tests.

TABLE 7
Geometric mean of EC₅₀ values across different assay parameters

| | Mol/L | ng/mL |
|---|---|---|
| Apremilast EC₅₀ mean of 8 | 2.87E−07 | 132 |
| determinations | | |
| Compound (I) EC₅₀ mean of 8 | 1.26E−08 | 6.43 |
| determinations | | |
| Roflumilast N-oxide EC₅₀ mean | 2.10E−08 | 8.80 |
| of 8 determinations | | |

Based on the mean EC₅₀ values shown in Table 7, the compound of formula (I) is 23 times more potent than apremilast based on molar concentrations and 21 times more potent based on ng/mL concentrations. The compound of formula (I) is 1.7 times (M)/1.4 times (ng/mL) more potent than Roflumilast.

### Example 5: Human Whole Blood Cytokine Secretion Assay

TruCulture blood collection and incubation tubes were tested with fresh human whole blood with or without addition of 20 nM of the compound of formula (I). The best results were obtained using heparin-stabilised blood and anti-CD3/CD28 test tubes. The secretion of IFN-γ, IL-1β, IL.-2, IL-4 and TNF-α was inhibited by the compound of formula (I) (Table 8), but IL-8 and IL-13 were not clearly inhibited.

TABLE 8
Inhibition of cytokines from whole blood

| % inhibition at | | | | | |
|---|---|---|---|---|---|
| 20 nM ^{a} | IFN-γ | IL-1 | IL-2 | IL-4 | TNF-α |
| Compound (I) | 54% | 40% | 50% | 44% | 60% |

^{a} Mean of 3 experiments.

The cytokine inhibition profile of the compound of formula (I) suggests that it will be beneficial for the treatment of inflammatory skin diseases. The compound potently inhibits the secretion of TNF-α and IL-1β, two cytokines that are highly associated with inflammation. The compound also inhibits IFN-γ, IL-2 and IL-4. IFN-γ is a T-cell derived Th1 cytokine that plays a role in Th1 immune responses, for example in HS.

### Example 6: PDE4 Inhibitors in the Chronic Oxazolone Model in BALB/cA Mice

The purpose of the study was to evaluate three different oral doses of the compound of formula (I) and the reference compound apremilast at one dose in the chronic oxazolone model.

### Materials and Methods

### Dosing Preparations

Four vials were delivered per treatment group (group 3-5). Every 3rd day, vehicle (1% methylcellulose) was added to the appropriate vials before dosing (see Table 9 below). The solution was stable for at least 7 days, but was made fresh every 3 days. Vials were delivered for treatment group 6. Every day vehicle (1% methylcellulose) was added just before dosing. It was ensured that the compound was dissolved completely before dosing. Dosing volume for all groups was 10 ml/kg or 0.01 ml/g bw.

TABLE 9
Dosing

| | | Dose | Vial | Vehicle | Final | |
|---|---|---|---|---|---|---|
| Group | Compound | (mg/kg bw) | (mg) | (ml) | concentration | |
| 3 | (1) | 10 | 9.6 | 9.6 | 1 | mg/ml |
| 4 | (I) | 3 | 3.0 | 10.0 | 0.3 | mg/ml |
| 5 | (I) | 1 | 0.9 | 9.0 | 0.1 | mg/ml |
| 6 | Apremilast | 30 | 10 | 3.33 | 3 | mg/ml |

Dexadresone was prepared each day by suspending 0.3 ml dexadresone vet (2 mg/ml) in 2.7 ml vehicle (1% methylcellulose) immediately before dosing. Final concentration was 0.2 mg/ml. The compound was fully suspended before dosing.

Animals used were female BALB/cABomTac mice, 7 weeks of age.

Mice in groups 2-7 were sensitized with oxazolone in acetone (0.8%) by applying 10 μl of the solution to each side of the right ear on day −7. Additionally, on day −7 mice in group 1 were dosed with 10 μl of acetone on each side of the left AND right ear. The 0.8% oxazolone solution is prepared as shown in Table 10 below:

TABLE 10
0.8% Oxazolone solutions

| No. of | Oxazolone | Acetone | |
|---|---|---|---|
| animals | (mg) | (ml) | Concentration |
| 50 | 12 | 1.5 | 0.80% |
| 60 | 14.4 | 1.8 | 0.80% |
| 70 | 16.8 | 2.1 | 0.80% |
| 80 | 19.2 | 2.4 | 0.80% |
| 90 | 21.6 | 2.7 | 0.80% |
| 100 | 24 | 3 | 0.80% |

One week after sensitization the mice in groups 2-7 were challenged for the first time with 0.4% oxazolone in acetone. Specifically, mice were dosed with 10 μl on each side of the right ear. Dosing was done at the following days: day 0, 3, 5, 7, 10, 12, 14, 16, 18 and 20. On the exact same days, mice in group 1 were dosed with 10 μl acetone on each side of the left AND right ear. The 0.4% oxazolone solution was prepared as shown in Table 11 below:

TABLE 11
0.4% Oxazolone solutions

| No. of | Oxazolone | Acetone | |
|---|---|---|---|
| animals | (mg) | (ml) | Concentration |
| 50 | 6 | 1.5 | 0.40% |
| 60 | 7.2 | 1.8 | 0.40% |
| 70 | 8.4 | 2.1 | 0.40% |
| 80 | 9.6 | 2.4 | 0.40% |
| 90 | 10.8 | 2.7 | 0.40% |
| 100 | 12 | 3 | 0.40% |

Oxazolone: 4-Ethoxymethylene-2-phenyl-2-oxazolin-5-one (≥90% (HPLC)), Sigma Aldrich, catalogue number E0753-5G, batch number 039K3533.

Mice were randomized according to the ear-thickness measured on day 10. The purpose was to create groups with similar mean ear thickness and standard deviations. The induced phenotype was treated orally with test compound as shown in Table 12 below. In group 2-6, 10 ml/kg bw of compound dissolved in methylcellulose (or methylcellulose only for group 2) was dosed orally once daily. Animals in group 1 were dosed orally with 10 ml/kg methylcellulose. All mice are treated from day 10 to day 21—both days included.

TABLE 12
Treatment schedule

| | No. of | | Concentration | Times | Volume p.o. | Dose |
|---|---|---|---|---|---|---|
| Group | animals | Compound | (mg/ml) | Daily | (ml/g bw) | (mg/kg) |
| 1 | 4 | Methylcellulose | NA | 1 | 0.01 | NA |
| 2 | 10 | Methylcellulose | NA | 1 | 0.01 | NA |
| 3 | 10 | Compound (I) | 1 | 1 | 0.01 | 10 |
| 4 | 10 | Compound (I) | 0.3 | 1 | 0.01 | 3 |
| 5 | 10 | Compound (I) | 0.1 | 1 | 0.01 | 1 |
| 6 | 10 | Apremilast | 3 | 1 | 0.01 | 30 |
| 7 | 10 | Dexadresone | 0.2 | 1 | 0.01 | 2 |

### Sampling

Blood and the right ear were sampled from all animals. Blood was sampled by cardiac puncture from animals anaesthetized with isoflurane (3% in oxygen). When the mice were fully anaesthetized (no interdigital reflex present), blood was sampled with a 25 G needle and a 1 ml syringe and transferred to 2.5 ml BD SST Vacutainer® tubes. After sampling vials were placed on ice for 30 minutes before being centrifuged for 10 min at 1000 G at 4° C. Immediately afterwards, serum was transferred to 1.4 ml noncoded u-bottom bulk Micronics tubes and stored at −80° C. Mice were euthanized by cervical dislocation immediately after blood sampling, while they were still fully anaesthetized.

Tissue from side A of the ear was placed in a Nunc cryotube and snap-frozen in liquid nitrogen. Samples were stored at −80° C. and/or sent to DMPK for analysis of drug concentration. Samples were taken 2 hours after the last application of treatment compound.

Tissue from side B of the ear was placed in a Nunc round-bottom cryotube and snap-frozen in liquid nitrogen. Samples were stored at −80° C. and/or sent to Molecular Biomedicine (Lili Rohde/Paola Lovato) for cytokine analysis. Samples were taken 2 hours after the last application of test compound.

Ear thickness was obtained online (directly into excel-spreadsheet) on day 10, 12, 14, 17, 19 and 21 with an Absolute Digimatic micrometer (Mitutoyo, Aurora, IL, ID-C1012CB code 543-274B). Right and left ears were measured on animals in group 1 and only the right ear was measured on animals from group 2 to 7. Ear measurements were performed before dosing, except on the day of termination where the ears were measured 2 hours after the last dosing. The relative ear-thickness was calculated by subtracting the mean ear-thickness of group 1 from the measured ear-thickness of animals in groups 2 to 7.

The compound concentration in serum was measured from animals in groups 2 to 6.

Statistical methods: One-way ANOVA with Dunnetts' post-test (compared to vehicle group=group 2); significance level 0.05.

### Results

As shown in FIG. 2, the ear thickness AUC of all dosed groups was significantly lower than AUC of the vehicle group. Blood was drawn from the animals 1 hour after the last dose at the expected Cmax. As shown in FIG. 3, the compound of formula (I) reached a similar reduction of ear thickness using a much lower dose (30-fold) and achieving much lower serum concentrations (>20 fold) compared to apremilast. Without being bound by theory, this could indicate that compound (I) may reach a similar efficacious outcome having 20-times lower systemic exposure and thus using a lower dose.

### Example 7: Emesis Study in Ferrets

The objectives of this study were to assess the emetogenic properties of the compound of formula (I) and roflumilast in the conscious, unrestrained ferret. Efficacy in the ferrets was measured as well, by analysing LPS-induced TNF-α ex vivo in whole blood after single oral administration. E. coli LPS (3 mg/kg) was injected ip 1.5 hours after oral administration of compounds, and TNF-α was measured 1 and 3 hours after LPS injection. TNF-α levels were determined in serum using a commercial ELISA kit.

The compounds were administered orally at doses of 1, 3, 10 and 30 mg/kg, using 1% (w/v) methylcellulose 400 centipoises in water as vehicle. Fasted animals were dosed and observed, with a minimum wash-out of 4 days between two sessions. After treatment, animals were singly housed and emetic episodes and associated prodromic signs (licking, mouth scratching, yawning, “wet dog” shakes, backward walking) were counted for approximately 3 hours. Following the emesis experiment blood samplings were performed at selected time points for serum clinical chemistry and pharmacodynamics.

### Results

The compound of formula (I) was found not to influence the health status or body weight gain of the ferrets. Emetogenic properties were absent at dose levels of 1 and 3 mg/kg and were observed at 10 and 30 mg/kg, in a dose-related manner. Therefore, the ‘no observable adverse effect level’ (NOAEL) for the compound of formula (I) in the conscious, unrestrained ferret is 3 mg/kg.

The compound of formula (I), administered at dose levels of 1, 3 and 10 mg/kg, attenuated, in a clear dose-related manner, the increase of TNF-α levels observed at 1 hour. A return towards baseline values was observed 3 hours after LPS administration.

Roflumilast, administered at dose levels of 1, 3, 10 and 30 mg/kg, did not influence the health status or body weight gain of the ferrets. Emetogenic properties were absent at the dose level of 1 mg/kg and were dose dependently observed at dose levels from 3 to 30 mg/kg. Therefore, the ‘no observable adverse effect level’ (NOAEL) for roflumilast in the conscious, unrestrained ferret is 1 mg/kg.

Roflumilast, administered at dose levels of 1 and 3 mg/kg, attenuated in a dose-related manner, the increase of TNF-α levels observed at 1 hour. A return towards baseline values was observed 3 hours after LPS administration.

Overall, the conclusion from this study is that the compound of formula (I) has a more favourable profile than Roflumilast, as the safety margin in ferrets is 3-fold higher than that of Roflumilast.

### Example 8: Clinical Trial Protocol, Phase 2

The following clinical trial may be performed using the compound of formula (I). Suitably the compound of formula (I) is orally administered to a subject in the form of a modified release composition, for example a modified release formulation described in WO 2020/148271, or as a composition described in Example 1 above.

The 10 mg and 30 mg orismilast tablet used in the clinical trial described in this example are modified release tablets described in Table 13.

TABLE 13

| | | | | | | | | Reference to |
|---|---|---|---|---|---|---|---|---|
| Name of | Quantity per tablet | | | | | | | Quality |
| Components | 10 mg | | 30 mg | | Placebo | | Function | Standard(s) |
| Drug Substance | | | | | | | | |
| Compound | 10 | mg | 30 | mg | — | | Drug | |
| of formula | | | | | | | substance | |
| (I) (orismilast) | | | | | | | | |
| Excipients | | | | | | | | |
| Lactose | 233 | mg | 213 | mg | 243 | mg | Filler | Ph. Eur. 0187 |
| monohydrate | | | | | | | | |
| Hypromellose | 52.5 | mg | 52.5 | mg | 52.5 | mg | Matrix former | Ph. Eur. 0348 |
| Silica, colloidal | 1.5 | mg | 1.5 | mg | 1.5 | mg | Glidant | Ph. Eur. 0434 |
| anhydrous | | | | | | | | |
| Magnesium stearate | 3.0 | mg | 3.0 | mg | 3.0 | mg | Lubricant | Ph. Eur. 0229 |
| Tablet core weight | 300 | mg | 300 | mg | 300 | mg | — | — |
| Film coating | | | | | | | | |
| Opadry ® II ^{Note 1} | 12 | mg | 12 | mg | 12 | mg | Film coating | |
| | | | | | | | system | |
| Coated | 312 | mg | 312 | mg | 312 | mg | — | — |
| tablet weight | | | | | | | | |

^{Note 1} Contain polyvinyl alcohol (Ph. Eur. 1961), macrogol (Ph. Eur. 1444), titanium dioxide (Ph. Eur. 0150), talc(Ph. Eur. 0438) and ferric oxide, yellow (NF).

### Objectives and Endpoints

### Primary Endpoint:

Percent change from Baseline in AN (abscesses and nodules) count at Week 16.

### Secondary Endpoints:

Change from Baseline in abscess, nodule, and draining fistula counts at Week 16.
Change from Baseline in IHS4 value at Week 16.
Change from Baseline in Patient's Global Assessment of Skin Pain (NRS) at Week 16.
Change from Baseline in HiS-QoL Total Score at Week 16

### Safety Endpoints:

Occurrence of treatment emergent adverse events (AEs).
Change in clinical laboratory parameters (haematology and biochemistry).
Change in vital signs.
Change in weight.

### Investigator Assessed Endpoints:

Patients achieving HiSCR at Week 16.
Patients achieving HiSCR 75 at Week 16.
Patients achieving HiSCR 90 at Week 16.
Patients achieving HS-PGA score of 0 or 1 at Week 16.
At least 50, 75, 90, or 100 percent reduction in total abscess and inflammatory nodule count overtime.
Change from baseline in high-sensitivity C-Reactive Protein (hs-CRP) over time.
HiSCR overtime.
Change from baseline in IHS4 value overtime.
Change in IHS4 category overtime.
HS-PGA score of 0 or 1 over time.

Use of rescue treatment (max 2 during active treatment, as needed during the follow-up).

### Patients Assessed Endpoints:

At least 30% reduction from baseline in Patient's Global Assessment of Skin Pain at Week 16 (NRS30).
Patient Global Assessment score overtime
Patients with flares at each visit and patients with at least one flare during treatment assessed at Week 16 (Flares defined as an at least 25% increase in AN counts with a minimum increase of 2 relative to Baseline).
Change from Baseline in DLQI Score at Week 16.
Change from Baseline in pruritus NRS overtime.
Change from Baseline in discharge NRS overtime.
At least 30% reduction from Baseline in Patient's Global Assessment of Skin Pain overtime (NRS30).
Change from baseline in HiSQOL Total Score overtime.
Change from baseline in Fatigue, DLQI, WPAI, McGill Pain, HADS, and EQ-5D.
Duration of remission after the end of IMP.
   Abbreviations: DLQI=Dermatology Life Quality Index; EQ-5D=European Quality of life-5 Dimensions; HADS=Hospital Anxiety and Depression Scale; HiSCR=Hidradenitis Suppurativa Clinical Response; HiSQOL=Hidradenitis Suppurativa Quality of Life; HS=Hidradenitis Suppurativa; Hs-CRP=High sensitivity C-Reactive Protein; HS-PGA=Physician Global Assessment; IHS4=International Hidradenitis Suppurativa Severity Score System; IMP=investigational medicinal product; NRS=numerical rating scale; WPAI=Work productivity and activity impairment.

### Overall Trial Design

This is a phase 2, single-centre, prospective, open-label, single-arm, exploratory clinical trial assessing the efficacy and safety of orismilast in patients with mild, moderate, or severe HS as defined by the IHS4 scores: A score of 3 or less signifies mild, a score of 4-10 signifies moderate and a score of 11 or higher signifies severe disease.

After signing the informed consent, and if all eligibility criteria are met, patients will be graded and assigned to a severity group. All groups will receive the same intervention.

Eligible patients will receive treatment with orismilast tablets twice daily for 16 weeks. The treatment dose will be according to a predefined titration scheme and adapted to the observed tolerance of each patient.

24 adult patients (men and women) will be included; 8 with mild, 8 with moderate, and 8 with severe HS.

The patients will enter into a 10-visit trial, including the screening and End-of-Study visit, 6 of which will be on-site visits, and 4 of which will be telephone consultations. The maximum duration of the trial, considering the screening phase of up to 4 weeks and a follow up phase of 14 weeks, is 34 weeks.

For each patient the duration of the trial will consist of 3 phases:
Screening phase (up to 4 weeks duration)
Treatment phase (16 weeks)
Follow up (14 weeks)
   An End of Study visit will be scheduled 14 weeks after last orismilast administration.

In addition to the standard site visit conducted at the trial site, this trial introduces telephone consultations (TC). These TCs will be conducted through a mandatory telephone call with the aim to reduce the burden of frequent site visits while ensuring close safety monitoring of patients.

The standard site visits are conducted at Screening, Baseline, Week 2, 8, 16 (End of Treatment) and 14 weeks after last IMP administration (End of Study). At each standard site visit the (sub)investigator will administer IMP including return and compliance check, as applicable.

The TCs are conducted at Week 1, 4, 12, and Follow-up (42 days after last drug administration). The patient will be contacted by the (sub)investigator who will collect assessments.

### Individual Phases

Screening phase: Prior to the performance of any trial related procedure, signed informed consent must be obtained from the patient. The screening phase will consist of 1 screening visit (Visit 1).

Treatment phase: The treatment phase will consist of an initial 2 weeks titration with progressive increase in dose from 10 mg BID to 30 mg BID (Week 0 [Visit 2], Week 1 [Visit 3; Telephone consultation], and Week 2 [Visit 4]) (“BID” means bis in die, i.e. twice per day).

At Week 2 (Visit 4) the investigator and patient will decide if further up-titration to reach 40 mg BID should be initiated. If further up-titration is initiated, the patient will increase the morning dose to 40 mg and maintain the evening dose at 30 mg during the next 2 days. At day 3 after the Week 2 visit, the dose will be increased to 40 mg BID and this dose will be maintained until the end of the treatment period (Week 16). During this part of the treatment phase, the patient will have a telephone consultation at Week 4 (Visit 5) and Week 12 (Visit 7) and will be required to return to the trial site at Week 8 (Visit 6) and Week 16 (Visit 8 [End of Treatment]).

Assessment of treatment efficacy will be undertaken using:
1. Investigator assessments:
   a. Counts of abscess, inflammatory nodule, and draining fistula at each on site visit day.
   b. International Hidradenitis Suppurativa Severity Score System (IHS4) at each on site visit day.
   c. Physician's Global Assessment of disease severity (HS-PGA) at each on site visit day.
2. Patient assessments
   a. Patients' Global Assessment of disease severity at Screening, Baseline, Week 2 (Visit 4), Week 8 (Visit 6), Week 12 (Visit 7), Week 16 (EoT, Visit 8), FU (Visit 9), and EoS (Visit 10).
   b. Completion of questionnaires covering skin related quality of life DLQI at Screening, Baseline, Week 2 (Visit 4), Week 8 (Visit 6), Week 12 (Visit 7), Week 16 (EoT), FU, and EoS.
   c. Completion of questionnaires covering HS specific quality of life (HiSQOL) at Screening, Baseline, Week 2 (Visit 4), Week 8 (Visit 6), Week 12 (Visit 7), Week 16 (EoT), FU, and EoS.
   d. Completion of questionnaire covering skin pain (Patient's Global Assessment of Skin Pain) at each visit day.
   e. Completion of questionnaire covering impact on work productivity (WPAI), anxiety and depression (HADS), fatigue (MFI-20), quality of life (EQ-5D), and McGill Pain questionnaire at Baseline, Week 16 (EoT), and EoS.

Safety and tolerability will be assessed by AE recording, vital signs (blood pressure, heart rate, body temperature), physical examination, ECG, and clinical laboratory (blood and urine).

### Biomarker Assessment Comprising

thermography of selected regions to assess temperature as a surrogate measure of inflammation and explore the methods utility as a biophysical biomarker.
ultrasound before and after treatment to evaluate target lesion identified clinically at baseline, and carefully described (location) and photographed to facilitate later recognition.
inflammatory biomarkers (CRP and neutrophilocyte/lymphocyte ratio)
skin biopsy before and after treatment to assess inflammatory markers (cytokines)
skin microbiome collected by simple skin swab.

In the case of early withdrawal from treatment before the Week 16 (Visit 8), the assessments scheduled for Visit 8 should be done at the end of treatment. Early withdrawal assessments should be performed as soon as possible.

Follow up phase: At Week 16, patients will roll-over to a no treatment observational period of up to Week 30. Conventional therapy will be started at the discretion of the patient. A follow up visit (TC) is scheduled 6 weeks after last IMP administration (Visit 9) and the End of Study visit (Visit 10) is scheduled 14 weeks after last IMP administration. For serious adverse events (SAEs), follow-up should continue until final outcome. In the case of early withdrawal from follow up before the End of Study visit (Visit 10) the assessments scheduled for Visit 10 should be done as soon as possible.

End of Trial Definition: The end of the trial is defined as the date of the last patient's last visit.

Trial Population: Prospective approval of protocol deviations to recruitment and enrolment criteria, also known as protocol waivers or exemptions, is not permitted. Adult male and female patients with mild to severe HS will be included in the trial if they fulfil all the inclusion criteria and do not present with any of the exclusion criteria.

### Inclusion Criteria:

Male or female adult patients, 18 years of age or older.
Have mild to severe HS for at least 1 year prior to the baseline visit, as determined by the investigator through participant interview and/or review of the medical history.
Have HS lesions in at least 2 distinct anatomic area (right/left axillary, inguinal, inframammary, abdominal, perineal).
Has a total inflammatory lesions (AN) count of greater than or equal to 2.
Total draining fistula count of less than or equal to 30.
A stable analgesic dose for 2 weeks prior to baseline.
Women of childbearing potential (WOCBP) must be ready and able to use highly effective methods of birth control per ICH M3 that result in a low failure rate of less than 1% per year when used consistently and correctly, for the duration of the trial and 16 weeks after last administration. A list of contraception methods meeting these criteria is provided in the patient information.
Signed and dated written informed consent in accordance with GCP and local legislation prior to the start of any screening procedures.

### Exclusion Criteria:

1. Presence of active skin lesions other than HS that could interfere with the assessment of HS.
2. Receipt of prescription topical therapies for HS within 7 days prior to the baseline visit (Visit 2).
3. Receipt of systemic therapies for HS, within 28 days prior to the baseline visit.
4. Any oral antibiotic within 28 days prior to baseline visit.
5. Receipt of a live vaccine within 14 days prior to screening.
6. Biologic use for indications other than HS within a minimum of 30 days or 5 half-lives of the drug, whichever is longer, prior to baseline.
7. Treatment with any investigational drug of chemical or biologic nature within a minimum of 30 days or 5 half-lives of the drug, whichever is longer, prior to baseline.
8. Patients who are immunosuppressed, or are taking medication that could have an immunosuppressive effect.
9. Women who are pregnant, nursing, or who plan to become pregnant while in the trial. Women who stop nursing before the study drug administration do not need to be excluded from participating.
10. Active systemic infection and/or fever—or clinical signs of local infection (stinging, increased soreness, burning sensation, erythematous perilesional halo or other signs of infection)—during the last 2 weeks prior to first drug administration.
11. History of allergy/hypersensitivity to the systemically administered trial medication agent or its excipients.
12. Patient with a transplanted organ (with exception of a corneal transplant >12 weeks prior to screening) or who have ever received stem cell therapy (e.g., Remestemcel-L).
13. Any documented active or suspected malignancy or history of malignancy within 5 years prior to the screening visit, except appropriately treated basal cell carcinoma of the skin, squamous cell carcinoma of the skin or in situ carcinoma of uterine cervix.
14. Relevant chronic or acute infections (exception: common cold), as determined by the investigator, including human immunodeficiency virus (HIV) or viral hepatitis. A patient can be re-screened if the patient was treated and is cured from the acute infection.
   a. In case of a positive hepatitis C antibody test, a positive reflex testing for Hepatitis C RNA PCR is considered positive.
15. Major surgery (major according to the investigator) performed within 12 weeks prior to first study drug administration or planned during the study (e.g. hip replacement, aneurysm removal, stomach ligation).
16. Severe, progressive, or uncontrolled hepatic disease, defined as >3-fold Upper Limit of Normal (ULN) elevation in AST or ALT or alkaline phosphatase, and >2-fold ULN elevation in alkaline phosphatase.
17. Evidence of a current or previous disease, medical condition (including chronic alcohol or drug abuse or any condition) other than HS, surgical procedure, psychiatric or social problems, medical examination finding (including vital signs and ECG), or laboratory value at the screening outside the reference range that in the opinion of the investigator is clinically significant and would compromise the safety of the patient or compromise the quality of the data, make the study participant unreliable to adhere to the protocol, comply with all study visits/procedures or to complete the trial.
18. Planned use of laser or other hair removal procedures over HS-affected areas during the trial period.
19. Planned HS surgery during the trial period.
20. Any condition for which the use of any prohibited medication (see Table 16) is indicated.

### Treatments Administered

Table 14 shows an example of an medicinal product that may be investigated using the trial:

TABLE 14
Identification of investigational medicinal product

| | |
|---|---|
| Study Treatment Name: | Orismilast modified release tablet |
| Dosage formulation: | Tablet |
| Unit dose strength(s)/ | 10 mg |
| Dosage level(s): | 30 mg |
| Active ingredient name | Orismilast (compound of formula (I)) |
| Excipients | See Table 1 |
| Study Treatment Name: | Orismilast modified release tablet |
| Route of Administration | oral |
| Dosing instructions: | Twice daily, morning and evening |
| | There should be at least 8 hours |
| | between the 2 administrations |
| Packaging and Labelling | Orismilast will be provided in |
| | alu-alu blister packaging with 10 |
| | tablets each |

Patients will be instructed to take tablets of IMP at home two times daily. One dose in the morning and one dose in the evening, starting from Day 1 onwards according to the dose titration scheme, Error! Reference source not found.5.

Patients will be provided with treatment instructions containing further guidance and precautions to follow when taking the IMP.

During the first 2 weeks there will be a progressive increase in dose until Week 2 (Visit 4) when the patient will be at 30 mg BID. At this visit the investigator and patient will decide if further up-titration to reach 40 mg BID should be initiated. If further up-titration is initiated, the patient will increase the morning dose to 40 mg and maintain the evening dose at 30 mg during the next 2 days. At day 3 after the Week 2 visit, the dose will be increased to 40 mg BID and this dose will be maintained until the end of the treatment period (Week 16).

In case of uncertainties or start of adverse events (AEs), the patient will primarily have telephonic access to a study-nurse. Should the patient wish to do so, or develop symptoms of AEs, the patient can discuss with the investigator the decrease of the daily dose to the last well tolerated dose. If this new dose is well tolerated, a new increase in the dose is left at the discretion of the patient and investigator.

TABLE 15
Dose titration scheme

| | Day | | | | | | | |
|---|---|---|---|---|---|---|---|---|
| | D 1 | D 1 | D 2 | D 2 | D 3 | D 3 | D 4 | D 4 |
| | AM | PM | AM | PM | AM | PM | AM | PM |
| Dose, | 10 | 10 | 10 + 10 | 10 | 10 + 10 | 10 + 10 | 10 + 10 | 10 + 10 |
| mg | | | | | | | | |

| | Day | | | | | | | | |
|---|---|---|---|---|---|---|---|---|---|
| | | | | | | | From | | From |
| | | | | | | | D 8 to | W 2 | W 2 |
| | | | | | | | W 2 | visit + | visit + |
| | D 5 | D 5 | D 6 | D 6 | D 7 | D 7 | visit | 1 & W 2 | 3 until |
| | AM | PM | AM | PM | AM | PM | AM/PM | visit + 2 | EoT |
| Dose, | 30 | 10 + 10 | 30 | 10 + 10 | 30 | 30 | 30/2 | 30 + 10/ | 30 + 10/ |
| mg | | | | | | | | 30 | 30 + 10 |

EOT = End of Treatment

Prohibited medications: Restricted medications are not allowed while the patient is on trial treatment and prior to visit 2 for durations as specified below, Table 16. Patients are allowed restricted medication in the follow-up period after the last treatment.

TABLE 16
Prohibited treatments/medications

| | Washout period prior to | |
|---|---|---|
| Medication | Visit 2 (baseline) | |
| Topical antibacterial agents for HS | 1 | week |
| Topical corticosteroids for HS | 1 | week |
| Systemic antibiotics¹ | 4 | weeks |
| Systemic corticosteroids | 4 | weeks |
| Biologics | 12 | weeks |
| Other systemic immunomodulatory agents | 4 | weeks |
| Live vaccines | 6 | weeks |
| Opioid analgesics² | 2 | weeks |
| Non-opioid analgesics³ | 1 | week |
| Spironolactone⁴ | 1 | week |
| Metformin⁴ | 1 | week |
| Topical cannabis oil⁵ | 1 | week |
| Tricyclic antidepressants (e.g., nortriptyline, | 4 | weeks |
| amitriptyline, imipramine, desipramine) | | |
| Type 1C antiarrhythmics (e.g. propafenone, | 4 | weeks |
| flecainide, encainide) | | |

¹Systemic antibiotics can be used for indications other than HS and for a duration of less than or equal to 28 days during the entire course of the trial. (until last treatment)
²Opiate analgesics restricted for HS and non-HS indications.
³Non-opioid analgesics for HS allowed for as needed use. Analgesics for indications other than HS only as needed.
⁴Restricted if used for HS. If used for non-HS indication, dose should be stable over 3 months prior to V2
⁵Restricted if used for HS/over affected areas. Other alternative therapies for HS are restricted, unless permitted after discussion with PI and trial team.

### Efficacy Assessments: Assessment of Treatment Efficacy Will be Undertaken Using:

### Investigator Assessments:

Counts of abscess, inflammatory nodule, and draining fistula. The lesions are defined in a Pan-European consensus paper (Daxhelet et al., Dermatology. 2020; 236(5):431-438). A nodule is defined as a palpable lesion, with a solid content, round rather than pointed, diameter >10 mm; usually deep seated, not raised; painful (spontaneously or on palpation) or not; inflammatory or not. An abscess is defined as a palpable lesion, with liquid content (pus), fluctuant, soft on palpation, diameter >10 mm, usually deep seated; acute, very painful phenomenon. A fistula is defined as one or several long, permeable channels connecting 2 (or more) suppurative cavities and/or skin openings, draining or not.
Hidradenitis Suppurativa Clinical Response (HiSCR): A treatment target based on correlation between the changes in lesion counts and PROM (pain and HRQoL). Hidradenitis Suppurativa Clinical Response (HiSCR; a 250% reduction from baseline in abscess and inflammatory nodule count and no increase in abscess or draining fistula counts) (Kimball et al., Ann Intern Med. 2012 Dec. 18; 157(12):846-55; Kimball et al., J Eur Acad Dermatol Venereol. 2016 June; 30(6):989-94). Subsequently modified for further differentiation: HiSCR75; a ≥75% reduction from baseline in abscess and inflammatory nodule count and no increase in abscess or draining fistula counts) and HiSCR-90; a ≥90% reduction from baseline in abscess and inflammatory nodule count and no increase in abscess or draining fistula counts)
International Hidradenitis Suppurativa Severity Score System (IHS4): A validated international clinimetric scale (Zouboulis et al., Br J Dermatol. 2017 November; 177(5):1401-1409). The score is based on a count of inflamed lesions. The resulting IHS4 score is arrived at by the number of nodules (multiplied by 1) plus the number of abscesses (multiplied by 2) plus the number of draining tunnels (multiplied by 4). A total score of 3 or less signifies mild, 4-10 signifies moderate and 11 or higher signifies severe disease.
Physician's Global Assessment of disease severity (HS-PGA): The six-point hidradenitis suppurativa Physician Global Assessment (PGA) ranges from clear to very severe (Kimball et al., Ann Intern Med. 2012 Dec. 18; 157(12):846-55). It is used in clinical trials to measure clinical improvement in inflammatory nodules, abscesses, and draining fistulae. The six stages are;
   Clear: No inflammatory or non-inflammatory nodules.
   Minimal: Only the presence of non-inflammatory nodules.
   Mild: Less than 5 inflammatory nodules or 1 abscess or draining fistula and no inflammatory nodules.
   Moderate: Less than 5 inflammatory nodules, or 1 abscess or draining fistula and 1 or more inflammatory nodules, or 2-5 abscesses or draining fistulas and less than 10 inflammatory nodules.
   Severe: 2-5 abscesses or draining fistulas and 10 or more inflammatory nodules.
   Very severe: More than 5 abscesses or draining fistulas.

### Patient Assessments:

Patients' Global Assessment of disease severity using an anchored (0=no disease, 10=worst imaginable disease severity) 0-10 Numerical Rating Scale (NRS).
Completion of questionnaire covering skin pain Patient's Global Assessment of Skin Pain (0=no pain, 10=worst imaginable pain) 0-10 Numerical Rating Scale (NRS).

In addition, patients will complete the following questionnaires:
Completion of questionnaires covering HS specific quality of life (HiSQOL).
Completion of questionnaires covering skin related quality of life DLQI.
Completion of questionnaires covering quality of life EQ-5D.
Completion of questionnaire covering pain McGill Pain questionnaire.
Completion of questionnaire covering impact on work productivity (WPAI).
Completion of questionnaire covering fatigue (MFI-20)
Completion of questionnaire covering anxiety and depression (HADS).
The Hidradenitis Suppurativa Quality of Life (HiSQOL): The HiSQOL is based on the work of the Hidradenitis SuppuraTiva cORe outcomes set International Collaboration (HISTORIC). HiSQOL has been developed and validated systematically and is a 17-item questionnaire that contains HS specific items such as drainage and odor in addition to more general skin specific items. HiSQOL is a HS-specific questionnaire designed to evaluate HRQOL in clinical trials (Thorlacius et al., Skin Appendage Disord. 2019 June; 5(4):221-229; Kirby et al., Br_J Dermatol. 2020 August; 183(2):340-348). It has a recall-period of 7 days and consists of 17 items divided into three domains: Four symptom questions, five psychosocial questions, and eight activities adaptation questions. For each item a score between 0 and 4 is given, with a higher score representing a greater adverse impact on HRQOL. The scores are summated for all items (maximum score: 68)1. Correlation with DLQI was 0.9, demonstrating very strong convergent validity.
Dermatology Life Quality Index (DLQI): A questionnaire of 10 questions concerning the patients' perception of the impact of skin diseases on different aspects of their health-related quality of life over the last week. Each question is scored on a four-point scale (0-3) resulting in a range of 0-30 points (0=Disease has no impact on quality of life, 30=Disease has maximum impact on quality of life). A validated scale first introduced by Finlay and Khan, Clin. Exp. Dermatol., 19 (1994), pp. 210-216).
European quality of life-5 Dimensions on a 5-level scale (EQ-5D-5L): An EQ-5D is a standardized instrument for measuring generic health status in terms of five dimensions: mobility, self-care, usual activities, pain/discomfort, and anxiety/depression. Each dimension receives a value of 1-5 leaving 55 55) different health states. The score is combined with an overall patient rating of health from 0-100 where 0 is worst imaginable health and 100 is best imaginable health. The scale was further developed from the original EQ-5D by Herdman et al., Qual Life Res. 2011 December; 20(10):1727-36.
McGill Pain questionnaire: This can be used to evaluate the sensation, strength and change over time of experienced pain. It can monitor pain over time or determine the effectiveness of intervention (Melzack, Pain: September 1975, Volume 1, Issue 3, p277-299).
Work Productivity and Activity Impairment Questionnaire: Specific Health Problem V2.0 (WPAI:SHP): A questionnaire describing work impairment due to a specific disease. Outcomes are expressed as impairment percentages, with higher numbers indicating greater impairment and less productivity (Reilly et al., Pharmacoeconomics. 1993 November; 4(5):353-65).
Anxiety and depression (HADS): A questionnaire comprising seven questions for anxiety and seven questions for depression. Each question is connected to four answers retrieving 0-3 points. For each condition, 0-7 points corresponds to normal case; 8-10 to borderline abnormal; and 11-21 to abnormal case (Zigmond and Snaith, Acta Psychiatrica Scandinavica (1983), 67(6): 361-370).
Multidimensional Fatigue Inventory 20 (MFI-20): The MFI-20 was invented by Smets et al., J Psychosom Res 1995; 39: 315-25. It consists of 20 items describing five subscales of fatigue: General Fatigue (GF), Physical Fatigue (PF), Reduced Motivation (RM), Reduced Activity (RA), and Mental Fatigue (MF). For each of the items the respondent must specify the extent to which the particular statements relate to him/her on a five-point scale, ranging from Yes, that is true to No, that is not true.

Biomarkers: Samples for biomarker research will be collected from all patients. The following biomarkers will be evaluated to explore possible differences in patients with mild, moderate, or severe HS and to evaluate their association with the observed clinical responses to orismilast:
Thermography of selected region.
Ultrasound of target lesion.
CRP (plasma).
Neutrophilocyte/lymphocyte ratio (plasma).
Inflammatory markers (cytokines) in skin (biopsies before and after treatment). Skin biopsies will be obtained as a standard 4 mm punch biopsy at day 0 and week 16. Samples will be stored for analyses (to be performed after the last visit of the last patient) in the biobank of Zeeland University Hospital, Roskilde and destroyed after analysis.
Skin microbiome before and after treatment by simple skin swab.

### Efficacy Analyses

### Analysis of Primary Efficacy Endpoint

The primary efficacy endpoint, percent change in AN count from baseline to Week 16, will be analysed for the full analysis set and the per protocol set.

AN count at each visit and for each severity group will be summarised. Change and percent change from baseline to Week 16 will also be included. For the Week 16 visit both observed data and completed data (with missing data imputed using LOCF imputation) will be presented.

The primary endpoint, percent change in AN count at Week 16, will be compared pair wise between severity groups using t-test supplemented by non-parametric Mann-Whitney U Test.

For the total sample the null hypothesis of no change from Baseline to week 16, H0: μ=0, will be tested using a one-sample t-test. The 95% confidence interval for the percentage change will be estimated

### Analysis of Secondary Efficacy Endpoints

All secondary efficacy endpoints will be analysed for the full analysis set.

Abscess, nodule, and draining fistula counts, IHS4, patients global assessment pain (NRS) and HiSQOL Total Score will be tabulated for each visit. The change from baseline to Week 16 will be included also.

The change from Baseline in abscess, nodule, and draining fistula counts, IHS4, patients global assessment pain (NRS), and HiSQOL Total Score to Week 16, will be compared pair wise between severity groups using t-test supplemented by non-parametric Mann-Whitney U Test.

For the total sample the null hypothesis of no change from Baseline to week 16, H0: μ=0, will be tested using a one-sample t-test. The 95% confidence interval for the percentage change will be estimated.

### Analysis of Other Endpoints Assessed by the Investigator and by Patients

All other endpoints will be summarized for the full analysis set. Tabulations of data may be supplemented by graphic displays. In general, no formal significance tests will be applied.

### Patients Achieving HiSCR at Week 16, Patients Achieving HiSCR75 at Week 16, and Patients Achieving HiSCR90 at Week 16

The number and percentage of patients who achieve each of these endpoints will be tabulated for each severity group. In addition, the number and percentage of patients who achieve HiSCR will be tabulated for each severity group at each visit.

For the total sample the null hypothesis of no response to treatment at week 16, H0: μ=0, will be tested using a binominal test. The 95% confidence interval for the percentage of patients achieving HiSCR will be estimated using the binominal distribution.

### Investigators Global Assessment of Disease Severity (HS-PGA)

The number and percentage of patients who achieve ‘treatment success’ according to HS-PGA (score 0 or 1) will be tabulated at each visit for each severity group.

Patients with at least 50%, 75%, 90%, or 100% reduction from baseline in total abscess and nodule count (AN count) overtime.

The number and percentage of subjects who achieve these end points will be tabulated at each visit for each severity group.

Change from baseline in High-Sensitivity C-Reactive Protein (hs-CRP) will be summarized for each severity group at each visit.

Patient's global assessment of disease severity and HiSQOL Total Score and change from Baseline will be summarized at each visit and for each severity group.

IHS4 score and change from baseline will be summarized at each visit and for each severity group.

Change in IHS4 category overtime.

The IHS4 category (mild, moderate, severe) will be tabulated for each visit. The change in category will for each visit be summarized for each severity group as proportion of patients changing status as worsening, no change and improving severity category.

Patients with at least 30% reduction from baseline in Patient's Global Assessment of Skin Pain over time. The number and percentage of subjects who achieve this end point will be tabulated at each visit for each severity group.

Change from baseline in pruritus and discharge as assessed by NRS will be summarized for each severity group at each visit.

### European Quality of Life-5 Dimensions (EQ-5D)

Each of the 5 dimensions ‘mobility’, self-care’, ‘usual activities’, ‘pain/discomfort’, and ‘anxiety/depression’, the index score, will be summarized at each visit and for each severity group.

### Dermatology Life Quality Index (DLQI)

The total DLQI score and change in DLQI will be summarized at each visit and for each severity group.

### Hospitality Anxiety and Depression Scale (HADS)

The total HADS score and change in HADS score will be summarized for each severity group at Baseline and Week 16.

### WPAI, McGill Pain, and Fatigue Assessed by MFI-20

The total score will be summarized for each severity group at Baseline and Week 16. The change from Baseline to Week 16 will be summarized also.

### Duration of Remission

The remission period is the time between EoT and relapse date, relapse date being defined as the earliest date where the patient would have lost at least 50% of the benefit he got during the treatment phase assessed be % change in AN count.

For those patients who experience a reduction in AN count from baseline to EoT the median remission time will be estimated using a Kaplan-Meier survival analysis.

Patients with Flares

A flare is determined as an at least 25% increase in AN counts with a minimum increase of 2 relative to Baseline.

The proportion of patients with flares will be tabulated at each visit for each severity group.

In addition, the proportion of patients who experienced at least one flare during treatment period from baseline to week 16 will be tabulated for each severity group.

### Example 9: Clinical, Phase 2—Initial Response of Orismilast (Compound of Formula (I)) Treatment of the First HS Patient from a Phase 2A Trial

### Methods: As Per Example 8

Patient: A 34 year old male with severe HS in axillae, groins and buttocks. The patient was a smoker since youth and there was no family history of HS, acne or IBD. The patient had been previously treated with tetracyclin, rifampicin-clindamycin, adalimumab, secukinumab, and iv. ertapenem with no or only temporary efficacy against the HS.

The patient received titration of orismilast from 10 mg twice daily to 30 mg twice daily in week 1 followed by 30 mg twice daily ongoing. Assessments were done at baseline, day 15 and day 57 of international HS severity score (IHS4), Visual analog scale of pain (VAS pain), HS specific quality of life (HiSQOL), Dermatology Life Quality Index (DLQI), physician's and patient's global assessment, and blood samples.

### Results

The patient experienced immediate clinical improvement with reduction of tissue edema and pain, as well as increased mobility. Reduction of disease activity from baseline to day 15 was universally perceptible and continued through to day 57 for IHS4, VAS pain, HiSQOL, DLQI: Table 17 and FIG. 4.

TABLE 17

| Time | IHS4 | VAS pain | HiSQOL | DLQI |
|---|---|---|---|---|
| Baseline | 65 | 9 | 54 | 25 |
| Day 14 | 61 | 7 | 42 | 23 |
| Day 57 | 62 | 6 | 46 | 22 |

Reductions in blood inflammatory biomarkers were also observed from baseline to day 57: C-reactive protein (89 vs. 84); erythrocyte sedimentation rate (94 vs. 88); leukocytes (14.0 to 11.5); and thrombocytes (402 vs. 365 (at day 14)).

The physician's global assessment of disease activity remained very severe. The patient's global assessment was very severe at day 15, but reduced to severe at day 57.

### CONCLUSIONS

Orismilast induced a rapid clinical and paraclinical improvement of inflammation in very severe HS in this patient, who was refractory to conventional HS therapies. Notably the patient experienced a rapid and marked reduction in swelling and pain associated with the HS.

### Example 10: Clinical, Phase 2—Initial Response of Orismilast Treatment of the First HS Patients from a Phase 2A Trial

### Methods: As Per Example 8

### Results

The trial is ongoing. The first four HS patients enrolled in the trial treated with orismilast in accordance with the protocol described in Example 8 exhibited initial signs of improvement in HS, including in patients that were non-responsive to prior biological HS treatments.

### Further Embodiments

The invention is further illustrated by the following numbered clauses:

P1. A compound of formula (I):

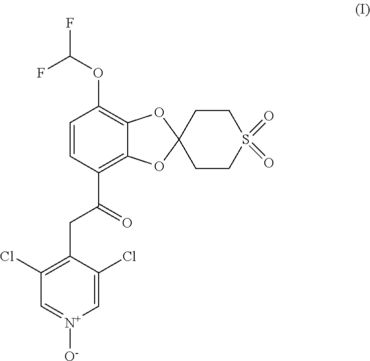 [SMILES: O=C(Cc1c(Cl)c[n+]([O-])cc1Cl)c1ccc(OC(F)F)c2c1OC1(CCS(=O)(=O)CC1)O2]

or a pharmaceutically acceptable salt, solvate or hydrate thereof, for use in the treatment of hidradenitis suppurativa (HS) in a subject.

P2. A compound of formula (I) as defined in clause P1, or a pharmaceutically acceptable salt, solvate or hydrate thereof, for use in preventing the progression of disease in a subject with mild or moderate HS.

P3. The compound for use of clause P1 or clause P2, wherein said treatment comprises oral, topical and/or intravenous administration of the compound of formula (I), or a pharmaceutically acceptable salt, solvate or hydrate thereof.

P4. The compound for use of any one of clauses P1 to P3, wherein the treatment is administered for at least 4 weeks, optionally at least 8 weeks.

P5. The compound for use of any one of clauses P1 to P4, wherein the treatment is administered for no more than 20 weeks, optionally no more than 16 weeks.

P6. The compound for use of any preceding clause, wherein the treatment comprises administering a total daily dose of no more than 120 mg, optionally no more than 80 mg.

P7. The compound for use of any preceding clause, wherein the treatment is administered twice daily.

P8. The compound for use of any preceding clause, wherein the treatment comprises administering a dose of from 10 to 60 mg, e.g. from 30 to 40 mg.

P9. The compound for use of any preceding clause, wherein the compound is comprised within a modified-release formulation.

P10. The compound for use of any preceding clause, wherein the compound is formulated for oral administration, optionally in the form of a tablet or capsule.

P11. The compound for use of any one of clauses P1 to P9, wherein the compound is formulated for topical administration.

P12. The compound for use of any preceding clause, wherein the subject is a human or animal, optionally wherein the subject is a human.

P13. The compound for use of any preceding clause, wherein the subject has mild HS.

P14. The compound for use of any one of clauses P1 to P12, wherein the subject has moderate HS.

P15. The compound for use of clause P1, or any of clauses P3 to P12 when dependent on clause 1, wherein the subject has severe HS.

P16. The compound for use of any preceding clause, wherein the subject is suffering from a comorbidity selected from obesity, metabolic syndrome, inflammatory bowel disease, spondyloarthropathy, or any combination thereof.

P17. The compound for use of any preceding clause, wherein the subject has not been previously treated with antibody therapy or a TNF-α inhibitor.

P18. The compound for use of any preceding clause, wherein the treatment is administered in combination with a further therapy.

P19. The compound for use of any preceding clause, wherein the further therapy is selected from an anti-androgenic agent, a hormone, an antibiotic (e.g. dapsone), a retinoid, an anti-inflammatory agent (including steroids, non-steroidal anti-inflammatory agents and colchicine), an analgesic, an immunosuppressive agent, an antibody, surgery, metformin, a nutritional supplement (e.g. zinc gluconate), a TNF-α inhibitor (e.g. adalimumab), and IL-1 inhibitor (e.g. anakinra), an anti-IL-17 drug (e.g. secukinumab), and anti-IL-23 drug (e.g. risankizumab), and anti-IL-12/23 drug (e.g. ustekinumab), a Janus Kinase (JAK) inhibitor (e.g. tofacitinib), or any combination thereof.

P20. The compound for use of any preceding clause, wherein said treatment provides selective inhibition of PDE4D and/or PDE4B, optionally wherein said treatment provides selective inhibition of PDE4D3 and/or PDE4B2.

1. A compound of formula (I):

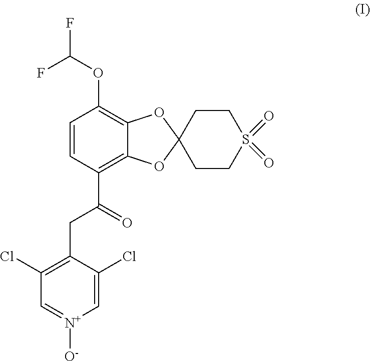 [SMILES: O=C(Cc1c(Cl)c[n+]([O-])cc1Cl)c1ccc(OC(F)F)c2c1OC1(CCS(=O)(=O)CC1)O2]

or a pharmaceutically acceptable salt, solvate or hydrate thereof, for use in the treatment of hidradenitis suppurativa (HS) in a subject.

2. A compound of formula (I) as defined in clause 1, or a pharmaceutically acceptable salt, solvate or hydrate thereof, for use in preventing the progression of disease in a subject with mild or moderate HS.

3. The compound for use of clause 1 or clause 2, wherein said treatment comprises oral, topical and/or intravenous administration of the compound of formula (I), or a pharmaceutically acceptable salt, solvate or hydrate thereof.

4. The compound for use of any one of clauses 1 to 3, wherein the treatment is administered for at least 4 weeks, optionally at least 8 weeks.

5. The compound for use of any one of clauses 1 to 4, wherein the treatment is administered for no more than 20 weeks, optionally no more than 16 weeks.

6. The compound for use of any preceding clause, wherein the treatment comprises administering a total daily dose of no more than 120 mg, optionally no more than 80 mg.

7. The compound for use of any preceding clause, wherein the treatment is administered twice daily.

8. The compound for use of any preceding clause, wherein the treatment comprises administering a dose of from 10 to 60 mg, e.g. from 30 to 40 mg.

9. The compound for use of any preceding clause, wherein the compound is comprised within a modified-release formulation.

10. The compound for use of any preceding clause, wherein the compound is formulated for oral administration, optionally in the form of a tablet or capsule.

11. The compound for use of any one of clauses 1 to 9, wherein the compound is formulated for topical administration.

12. The compound for use of any preceding clause, wherein the subject is a human or animal, optionally wherein the subject is a human.

13. The compound for use of any preceding clause, wherein the subject has mild HS.

14. The compound for use of any one of clauses 1 to 12, wherein the subject has moderate HS.

15. The compound for use of clause 1, or any of clauses 3 to 12 when dependent on clause 1, wherein the subject has severe HS.

16. The compound for use of any preceding clause, wherein the subject is suffering from a comorbidity selected from obesity, metabolic syndrome, inflammatory bowel disease, spondyloarthropathy, or any combination thereof.

17. The compound for use of any preceding clause, wherein the subject has not been previously treated with biological therapy for HS (for example, an antibody therapy or a TNF-α inhibitor).

18. The compound for use of any one of clauses 1 to 16, wherein the subject is non-responsive or refractory to biological therapy for HS.

19. The compound for use of any preceding clause, wherein the treatment is administered in combination with a further therapy.

20. The compound for use of any preceding clause, wherein the further therapy is selected from: from an anti-androgenic agent, a hormone, an antibiotic (e.g. dapsone), a retinoid, an anti-inflammatory agent (including steroids, non-steroidal anti-inflammatory agents and colchicine), an analgesic, an immunosuppressive agent, an antibody, surgery, metformin, a nutritional supplement (e.g. zinc gluconate), a biological therapy for HS (e.g. a TNF-a inhibitor (e.g. adalimumab), an IL-1 inhibitor (e.g. anakinra), an anti-IL-17 drug (e.g. secukinumab), an anti-IL-23 drug (e.g. risankizumab), or an anti-IL-12/23 drug (e.g. ustekinumab)), a complement C5a inhibitor (e.g. avacopan), a Janus Kinase (JAK) inhibitor (e.g. tofacitinib, INCB054707 or upadacitinib), a leukotriene A4 hydrolase inhibitor (e.g. LYS 006), an IRAK4 degrader (e.g. KT-474), a IRAK4 inhibitor (e.g. PF-06650833), a tyrosine kinase 2 (TYK2) inhibitor (e.g. PF-06826647) or a TYK2/JAK1 inhibitor (e.g. PF-06700841), or any combination thereof.

21. The compound for use of any preceding clause, wherein said treatment provides selective inhibition of PDE4D and/or PDE4B, optionally wherein said treatment provides selective inhibition of PDE4D3 and/or PDE4B2.

1. A method of treating hidradenitis suppurativa (HS), the method comprising administering to a subject in need thereof a therapeutically effective amount of a compound of formula (I): 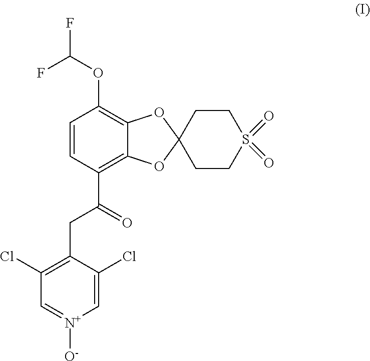 [SMILES: O=C(Cc1c(Cl)c[n+]([O-])cc1Cl)c1ccc(OC(F)F)c2c1OC1(CCS(=O)(=O)CC1)O2]
or a pharmaceutically acceptable salt, solvate or hydrate thereof.

2. The method according to claim 1, wherein the treatment prevents the progression of disease in a subject with mild or moderate HS.

3. The method according to claim 1, wherein the treatment reduces pain caused by or associated with HS.

4. The method according to claim 1, wherein the treatment reduces inflammation caused by or associated with HS.

5. The method according to claim 1, wherein said treatment comprises oral, topical and/or intravenous administration of the compound of formula (I), or a pharmaceutically acceptable salt, solvate or hydrate thereof.

6. The method of claim 1, wherein the compound is administered for at least 4 weeks.

7. The method of claim 1, wherein the compound is administered for no more than 20 weeks.

8. The method of claim 1, wherein the treatment comprises administering the compound of formula (I) in a total daily dose of no more than 120 mg.

9. The method of claim 1, wherein the compound is administered twice daily.

10. The method of claim 1, wherein the treatment comprises:
(i) administering the compound in a dose of 30 mg twice per day; or
(ii) administering the compound in a dose of 40 mg twice per day.

12. The method according to claim 1, wherein the compound is administered orally.

13. The method of claim 1, wherein the compound is comprised within a modified-release formulation.

14. The method of claim 13, wherein the modified-release formulation releases a mean amount of from about 11% to about 65% of the compound of formula (I) after 45 minutes and more than 80% of the compound of formula (I) after 180 minutes when placed in a dissolution medium of 900 ml 0.5% sodium dodecyl sulfate in 0.1N HCl using Ph. Eur. 2.9.3 Apparatus II and a paddle speed of 75 rpm.

15. The method of claim 13, wherein the modified-release formulation comprises formula (I), or a pharmaceutically acceptable salt, solvate or hydrate thereof and a hydrophilic matrix former.

16. The method of claim 13, wherein the modified-release formulation comprises the compound of formula (I), or a pharmaceutically acceptable salt, solvate or hydrate thereof; and 15% w/w to 25% w/w of hydroxypropyl methyl cellulose.

17. The method of claim 1, wherein the compound is formulated for oral administration.

18. The method of claim 1, wherein the compound is formulated for topical administration.

19. The method of claim 1, wherein the subject is a human.

20. The method of claim 1, wherein the subject has mild HS.

21. The method of claim 1, wherein the subject has moderate HS.

22. The method of claim 1, wherein the subject has severe HS.

23. The method of claim 1, wherein the subject is suffering from a comorbidity selected from obesity, metabolic syndrome, inflammatory bowel disease, spondyloarthropathy, or any combination thereof.

24. The method of claim 1, wherein the subject has not been previously treated with biological therapy for HS (for example, an antibody therapy or a TNF-α inhibitor).

25. The method of claim 1, wherein the subject is non-responsive or refractory to biological therapy for HS.

26. The method of claim 1, wherein the treatment is administered in combination with a further therapy for HS.

27. The method of claim 26, wherein the further therapy for HS is selected from: from an anti-androgenic agent, a hormone, an antibiotic, a retinoid, an anti-inflammatory agent, an analgesic, an immunosuppressive agent, an antibody, surgery, metformin, a nutritional supplement, a biological therapy for HS, a complement C5a inhibitor, a Janus Kinase (JAK) inhibitor, a leukotriene A4 hydrolase inhibitor, an IRAK4 degrader, a IRAK4 inhibitor, a tyrosine kinase 2 (TYK2) inhibitor or a TYK2/JAK1 inhibitor, or any combination thereof.

28. The method of claim 1, wherein said treatment provides selective inhibition of PDE4D and/or PDE4B.

29. The method of claim 14 wherein the hydrophilic matrix former comprises hydroxypropyl methyl cellulose.

30. The method of claim 14, wherein the modified-release formulation comprises the compound of formula (I), or a pharmaceutically acceptable salt, solvate or hydrate thereof; and 15% w/w to 25% w/w of hydroxypropyl methyl cellulose.

31. The method of claim 30, wherein the modified release formulation is in the form of a tablet.
